(19) United States
(12) Patent Application Publication
(10) Pub. No.: US 2025/0012909 A1
(43) Pub. Date: Jan. 9, 2025

(54) SYSTEMS AND METHODS FOR EVENT-DRIVEN ULTRASOUND SAMPLING

(71) Applicant: Luma Vision Limited, Dublin (IE)

(72) Inventors: Lydia Frances Neary-Zajiczek, Tralee (IE); Cédric Assambo, Dublin (IE); Stefan Wörz, Munich (DE); Christoph Hennersperger, München (DE)

(21) Appl. No.: 18/763,091

(22) Filed: Jul. 3, 2024

Related U.S. Application Data
(60) Provisional application No. 63/525,274, filed on Jul. 6, 2023.

(51) Int. Cl. G01S 7/52; A61B 8/00; A61B 8/08; A61B 8/12; B06B 1/02; G01S 15/89

(52) U.S. Cl. CPC G01S 7/52085; A61B 8/12; A61B 8/4488; A61B 8/483; A61B 8/546; A61B 8/58; G01S 15/8993; B06B 1/02

(57) ABSTRACT

The invention generally relates to ultrasound imaging, and, more particularly, to systems and methods for providing event-driven ultrasound imaging.

### CROSS-REFERENCE TO RELATED APPLICATIONS

This application claims priority to, and the benefit of, U.S. Provisional Application No. 63/525,274, filed Jul. 6, 2023, the content of which is incorporated by reference herein in its entirety.

### FIELD OF THE INVENTION

The invention generally relates to ultrasound imaging, and, more particularly, to systems and methods for providing event-driven ultrasound imaging.

### BACKGROUND

Ultrasound imaging is a medical imaging technique for imaging organs and soft tissues in a human body. An ultrasound image is produced based on the reflection of high-frequency sound waves off of body structures. The strength (amplitude) of the sound signal in conjunction with the time it takes for the wave to travel through the body provides the information necessary to produce the image.

Ultrasound imaging can help a physician evaluate, diagnose and treat various medical conditions. When making a diagnosis based on an ultrasound examination, physicians must rely on adequate image quality, acquisition of proper views, and sufficient quantification of all relevant structures and flows.

For example, catheter-based endovascular ultrasound imaging technology employed within the vasculature (e.g., intravascular ultrasound (IVUS) or intracardiac echocardiography (ICE)) is commonly performed with two-dimensional (2D)-ultrasound imaging. In IVUS/ICE imaging systems, an ultrasonic transducer assembly is attached to a distal end of a catheter. The catheter is carefully maneuvered through a patient's body to an area of interest, such as within a coronary artery (for the case of IVUS), or within the right atrium (for the case of ICE). The transducer assembly transmits ultrasound waves and receives echoes from those waves. The received echoes are then converted to electrical signals and transmitted to processing equipment, in which a resulting ultrasound image of the area of interest may be displayed.

In typical ultrasound systems configured to visualize inner body regions, dynamic forces are often employed, resulting in a dynamic movement of the body regions over time. These dynamic forces and movements make it difficult to stabilize internal imaging devices and to generate consistent and accurate images if imaging of the structure cannot be enabled in real-time (e.g., >20 Hz). As a result, the captured images often lack the necessary quality required to prescribe appropriate treatment or therapy. Because of the dynamic forces and movements in play, internal real-time imaging is limited to small two-dimensional areas or limited three-dimensional volumetric regions respectively. Further, difficult engineering tradeoffs exist between system complexity and achievable image quality, and the resources required for imaging. Thus, the final quality of the image obtained through ultrasound scanning is limited by the technical specifications of the equipment, the propagation of ultrasonic waves through the tissue analyzed, and the method used to reconstruct the images.

### SUMMARY

The present invention recognizes the limitations of current ultrasound imaging systems, particularly the limitations of resources necessary to provide the sustainable data rate required for imaging. The systems and methods of the invention use one or more novel algorithms to reduce the required average sampling rate for ultrasonic receive data, particularly in the context of digitalization at the tip of a catheter ultrasound device where available space is heavily constrained. In particular, the invention provides systems and methods for ultrasound image sampling that optimize the resources required for the acquisition and transfer of meaningful information to an external processing system. By sampling only meaningful information, the average data rate transmission requirement is reduced to minimize the overall power consumption and heat dissipation within the imaging device, as well as providing optimized interconnect assemblies between different modules of the signaling chain.

The systems and methods of the invention exploit properties of the ultrasound data generated by sampling only when the received data has changed sufficiently according to a previously defined threshold, which may be classified as an “event.” This threshold may be, for example, determined based on the clinical relevance of the structures being imaged and the characteristics of the received data. In this way, structures that are clinically relevant are sampled at a high data rate, while other anatomical areas are not sampled at a high data rate, thus reducing overall power consumption and heat dissipation within the imaging device. Systems and methods of the invention include novel event-driven architectures utilizing novel sampling algorithms capable of sampling beyond the well-defined level crossings at set voltage levels of prior art sampling architectures.

Accordingly, the invention provides improved imaging to minimize data transmission rate and maximize clinically relevant image quality, particularly in catheter-based ultrasound imaging.

Aspects of the invention provide systems for image sampling that include a hardware processor coupled to non-transitory, computer-readable memory containing instructions executable by the processor to cause the processor to receive data from an imaging device and run an event-driven sampling algorithm. The event-driven sampling algorithm causes the processor to analyze the received data to identify one or more characteristics associated with the received data and further detect the occurrence of one or more events. The one or more events include a change in the one or more characteristics as compared to a defined threshold for the one or more characteristic. Further, the event-driven algorithm causes the processor to tune a sampling rate of the imaging device based on a detected occurrence of the one or more events. In some embodiments, the defined threshold is determined based, at least in part, on a clinical relevance of a structure to be imaged and/or one or more characteristics of the received data.

In some embodiments, the received data comprises digitized voltage signals.

Accordingly, the processor, in some embodiments, is configured to access raw digitized voltage signals and tune one or more sampling parameters to minimize a data transmission rate and to maximize an image quality of a clinically relevant image. The defined threshold is a digitized voltage encoded at a lower bit depth than a full sampling bit depth, some embodiments. In some embodiments, the defined threshold is a change in output voltage by a defined parameter relative to a previous output voltage. For example, the image sampling by the imaging device is triggered at a full sampling rate for a set time for each occurrence of an event associated with the output voltage changing by the defined parameter relative to the previous output voltage, in some embodiments. In particular embodiments, the defined threshold is a change in output voltage by a set parameter in a previously stored threshold value, wherein the threshold value is updated and stored when image sampling by the imaging device is triggered. Further, in some embodiments, the defined threshold is one or more logarithmically spaced voltage levels.

In other embodiments, the received data comprises analog voltage signals. In some embodiments, the defined threshold is a change in an amplitude signal by a set parameter in a previously stored threshold amplitude signal, wherein only amplitude and phase of a carrier wave are sampled with the phase sampled concurrently with the amplitude and encoded at a lower bit rate than 16 bits. For example, the amplitude and the phase of the carrier wave are extracted via I/Q demodulation, in some embodiments.

In particular embodiments, the imaging device comprises a transducer comprising an array of individual imaging elements. For example, the transducer comprises a micro-electromechanical systems (MEMS)-based micromachined ultrasonic transducer configured as a two-dimensional (2D) array structure, in some embodiments. In other embodiments, the imaging elements are acoustic sensors activated by the processor to transmit and/or receive a plurality of incident acoustic wave signals as wave data. For example, the wave data comprises at least one of plane wave data and diverging wave data associated with one or more wave transmit-receive cycles carried out by the imaging elements. The wave data is full circumferential, three-dimensional (3D) image data, in some embodiments. Further, in some embodiments, the imaging device comprises a catheter-based ultrasound imaging device configured to transmit ultrasound pulses to, and receive echoes of the ultrasound pulses from, intravascular and/or intracardiac tissue.

In particular embodiments, the imaging device is a minimally invasive implantable device. Specifically, tuning of the sampling rate reduces an overall power consumption and heat dissipation of the device. In some embodiments, tuning of the sampling rate results in reduction of an average sampling rate of received data from the imaging device.

In some embodiments, the processor is embedded as part of an application-specific integrated circuit (ASIC).

In another aspect, the invention provides methods for image sampling. The method includes providing a hardware processor coupled to non-transitory, computer-readable memory containing instructions executable by the processor to cause the processor to receive data from an imaging device and run an event-driven sampling algorithm. The event-driven algorithm causes the processor to analyze the received data to identify one or more characteristics associated with the received data and further detect the occurrence of one or more events, wherein the one or more events comprises a change in the one or more characteristics as compared to a defined threshold for the one or more characteristic, and tune a sampling rate of the imaging device based on a detected occurrence of the one or more events.

In some embodiments of the method, the received data comprises digitized voltage signals. For example, the processor is configured to access raw digitized voltage signals, and tune one or more sampling parameters to minimize a data transmission rate and to maximize an image quality of a clinically relevant image, in some embodiments. In particular embodiments of the methods, the defined threshold is a digitized voltage encoded at a lower bit depth than a full sampling bit depth. The defined threshold is a change in output voltage by a defined parameter relative to a previous output voltage, in some embodiments. For example, the image sampling by the imaging device is triggered at a full sampling rate for a set time for each occurrence of an event associated with the output voltage changing by the defined parameter relative to the previous output voltage. Further, in some embodiments of the methods, the defined threshold is a change in output voltage by a set parameter in a previously stored threshold value, wherein the threshold value is updated and stored when image sampling by the imaging device is triggered. In particular embodiments, the defined threshold is one or more logarithmically spaced voltage levels.

In some embodiments of the methods, the received data comprises analog voltage signals.

In various embodiments of the methods, the defined threshold is a change in an amplitude signal by a set parameter in a previously stored threshold amplitude signal, wherein only amplitude and phase of a carrier wave are sampled with the phase sampled concurrently with the amplitude and encoded at a lower bit rate than 16 bits. For example, in some embodiments, the amplitude and the phase of the carrier wave are extracted via I/Q demodulation.

In some embodiments of the methods, the imaging device comprises a transducer comprising an array of individual imaging elements. Further, the transducer comprises a micro-electromechanical systems (MEMS)-based micromachined ultrasonic transducer configured as a two-dimensional (2D) array structure, in some embodiments. The imaging elements are acoustic sensors activated by the processor to transmit and/or receive a plurality of incident acoustic wave signals as wave data. For example, in some embodiments, the wave data comprises at least one of plane wave data and diverging wave data associated with one or more wave transmit-receive cycles carried out by the imaging elements. In some embodiments, the wave data is full circumferential, three-dimensional (3D) image data. In particular embodiments, the imaging device comprises a catheter-based ultrasound imaging device configured to transmit ultrasound pulses to, and receive echoes of the ultrasound pulses from, intravascular and/or intracardiac tissue. In some embodiments, the imaging device is a minimally invasive implantable device.

Further, in some embodiments, tuning of the sampling rate reduces an overall power consumption and heat dissipation of the device.

In some embodiments of the methods, the sampling rate results in reduction of an average sampling rate of received data from the imaging device.

In some embodiments of the methods of the invention, processor is embedded as part of an application-specific integrated circuit (ASIC).

### BRIEF DESCRIPTION OF THE DRAWINGS

FIGS. 1A and 1B are diagrammatic illustrations of an exemplary ultrasound system which systems and methods of the invention may be used.

FIG. 2 is a perspective view of an imaging catheter with which systems and methods of the invention may be used.

FIG. 3 illustrates a system according to one embodiment of the invention.

FIG. 4 is a block diagram of a method for providing event-driven sampling according to one embodiment of the invention.

FIGS. 5A and 5B show example results comparing an original image and the image determined using a differential algorithm according to one embodiment of the invention.

FIG. 6 is a graph showing experimental results including the average mean squared error for the differential algorithm, the difference threshold algorithm, the moving threshold algorithm and the triggered burst algorithm (using a burst length of 5 and 25), per bit level/log2 (Delta) using synthetic data.

FIG. 7 is a graph illustrating experimental results for the average transmission rate (%) for the differential algorithm, the difference threshold algorithm, the moving threshold algorithm and the triggered burst algorithm (using a burst length of 5 and 25), per bit level/log2 (Delta) using synthetic data.

FIG. 8 is a graph illustrating experimental results of the average mean squared error (Pre-Clinical Data (2022-07-28_IMMR)) for the differential algorithm, the difference threshold algorithm, the moving threshold algorithm and the triggered burst algorithm (using a burst length of 5 and 45), per bit level/log2 (Delta), using data from anatomical imaging in RA.

FIG. 9 is a graph showing experimental results for the average transmission rate (%) (Pre-Clinical Data (2022-07-28_IMMR)) for the differential algorithm, the difference threshold algorithm, the moving threshold algorithm and the triggered burst algorithm (using a burst length of 5 and 45), per bit level/log2 (Delta), using data from anatomical imaging in RA.

FIG. 10 is a graph illustrating the average mean squared error for the differential algorithm, the logarithmic algorithm, the difference threshold algorithm, the moving threshold algorithm, the triggered burst algorithm (burst length 5), the triggered burst algorithm (burst length 45) and the quadratic phase algorithm (bit level 3), per bit level/log2 (Delta), using pullback sequence.

FIG. 11 is a graph illustrating the average transmission rate (%) for the differential algorithm, the logarithmic algorithm, the difference threshold algorithm, the moving threshold algorithm, the triggered burst algorithm (burst length 5), the triggered burst algorithm (burst length 45) and the quadratic phase algorithm (bit level 3), per bit level/log2 (Delta) using pullback sequence.

FIG. 12 illustrates the data transmission histogram for the DIFFERENTIAL algorithm with delta/bit level 6.

FIG. 13 illustrates the data transmission histogram for the DIFFERENCE_THRESHOLD algorithm with delta/bit level 6.

FIG. 14 illustrates the data transmission histogram for the MOVING_THRESHOLD algorithm with delta/bit level 6.

FIG. 15 illustrates the data transmission histogram for the LOGARITHMIC_bit_level_6 algorithm with delta/bit level 6.

FIGS. 16A and 16B illustrate the average mean squared error and average transmission rate (%) versus Bit Level/log2 (Delta) using a sampling rate of 0.6 for the Differential, Logarithmic, and Moving Threshold algorithms.

FIGS. 17A and 17B illustrate the average mean squared error and average transmission rate (%) versus Bit Level/log2 (Delta) using a sampling rate of 1.0 for the Differential, Logarithmic, and Moving Threshold algorithms.

FIG. 18 illustrates graphs of mean data transmission rate (%), mean squared error, peak signal-to-noise ratio, and normalized root mean squared error versus bit_level for DIFFERENTIAL_f_sample_1000e-3.

FIG. 19 illustrates graphs of mean data transmission rate (%), mean squared error, peak signal-to-noise ratio, and normalized root mean squared error versus bit_level for DIFFERENTIAL_f_sample_800e-3.

FIG. 20 illustrates graphs of mean data transmission rate (%), mean squared error, peak signal-to-noise ratio, and normalized root mean squared error versus bit_level for DIFFERENTIAL_f_sample_600e-3.

FIG. 21 illustrates graphs of mean data transmission rate (%), mean squared error, peak signal-to-noise ratio, and normalized root mean squared error versus bit_level for LOGARITHMIC_f_sample_1000e-3.

FIG. 22 illustrates graphs of mean data transmission rate (%), mean squared error, peak signal-to-noise ratio, and normalized root mean squared error versus bit_level for LOGARITHMIC_f_sample_800e-3.

FIG. 23 illustrates graphs of mean data transmission rate (%), mean squared error, peak signal-to-noise ratio, and normalized root mean squared error versus bit_level for LOGARITHMIC_f_sample_600e-3.

FIG. 24 illustrates graphs of mean data transmission rate (%), mean squared error, peak signal-to-noise ratio, and normalized root mean squared error versus delta (bit_level) for MOVING_THRESHOLD_f_sample_1000e-3.

FIG. 25 illustrates graphs of mean data transmission rate (%), mean squared error, peak signal-to-noise ratio, and normalized root mean squared error versus delta (bit_level) for MOVING_THRESHOLD_f_sample_800e-3.

FIG. 26 illustrates graphs of mean data transmission rate (%), mean squared error, peak signal-to-noise ratio, and normalized root mean squared error versus delta (bit_level) for MOVING_THRESHOLD_f_sample_600e-3.

FIG. 27 includes graphs of mean data transmission rate (%), mean squared error, peak signal-to-noise ratio, and normalized root mean squared error versus bit_level for DIFFERENTIAL_f_sample_1000e-3, over all events.

FIG. 28 includes graphs of mean data transmission rate (%), mean squared error, peak signal-to-noise ratio, and normalized root mean squared error versus bit_level for DIFFERENTIAL_f_sample_800e-3, over all events.

FIG. 29 includes graphs of mean data transmission rate (%), mean squared error, peak signal-to-noise ratio, and normalized root mean squared error versus bit_level for DIFFERENTIAL_f_sample_600e-3, over all events.

FIG. 30 includes graphs of mean data transmission rate (%), mean squared error, peak signal-to-noise ratio, and normalized root mean squared error versus bit_level for LOGARITHMIC_f_sample_1000e-3, over all events.

FIG. 31 includes graphs of mean data transmission rate (%), mean squared error, peak signal-to-noise ratio, and normalized root mean squared error versus bit_level for LOGARITHMIC_f_sample_800e-3, over all events.

FIG. 32 includes graphs of mean data transmission rate (%), mean squared error, peak signal-to-noise ratio, and normalized root mean squared error versus bit_level for LOGARITHMIC_f_sample_600e-3, over all events.

FIG. 33 includes graphs of mean data transmission rate (%), mean squared error, peak signal-to-noise ratio, and normalized root mean squared error versus delta (bit_level) for MOVING_THRESHOLD_f_sample_1000e-3, over all events.

FIG. 34 includes graphs of mean data transmission rate (%), mean squared error, peak signal-to-noise ratio, and normalized root mean squared error versus delta (bit_level) for MOVING_THRESHOLD_f_sample_800e-3, over all events.

FIG. 35 includes graphs of mean data transmission rate (%), mean squared error, peak signal-to-noise ratio, and normalized root mean squared error versus delta (bit_level) for MOVING_THRESHOLD_f_sample_600e-3, over all events.

### DETAILED DESCRIPTION

The present invention recognizes the limitations of current tissue analysis and visualization using ultrasound technology, namely the required average sampling rate of ultrasonic receive data. The invention provides systems and methods to reduce the required average sampling rate of ultrasonic receive data.

For example, the sustainable data rate required for imaging is greater than 70.0 Gbit/s (8.86 GB/s). In particular, the digitized (16-bit) output voltage signal of a piezoelectric transducer corresponds to a received ultrasound wave and has the form of a sinusoid oscillating at a carrier frequency f_{c} that is modulated by the signal corresponding to the absorption and reflection of acoustic pressure by the environment being imaged. The signal is sampled at a sampling frequency fₛ, which for a 128-element transducer, fₛ=50 MHZ, imaging depth d=70 mm (4544 samples), and a Tx pulse repetition frequency f_{Tx}=10 KHz corresponds to a data rate of >70.8 Gb/s (86.6 Gb/s). Accordingly, the invention provides systems and architectures that include novel sampling algorithms to provide event-driven ultrasound image sampling.

Prior art event-driven sampling architectures primarily involve only well-defined level crossings at set voltage levels. The oscillatory nature of ultrasound data means that these defined architectures are inappropriate. This is because level crossing can occur even when clinically relevant data is not present, for example when imaging a blood pool with low level oscillations. In contrast, the present invention provides a flexible system that allows the systems and methods to directly access the raw digitized signals and tune the specific event-driven sampling parameters-for example a change in parameter(s)-to minimize data transmission rate and maximize clinically relevant image quality.

Accordingly, the invention provides sampling schemes that take advantage of the characteristics of the data to be sampled. The invention provides opportunities for optimization of the resources required for the acquisition and transfer of meaningful information to an external processing system. By only sampling meaningful information, the average data rate transmission requirement is reduced to minimize the overall power consumption and heat dissipation within, for example, a catheter ultrasound imaging device.

Systems of the invention may be operably connected with an ultrasound system with certain hardware and software for providing image reconstruction and imaging assembly control, for example as described in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein. The data may be processed using imaging protocols to extract anatomical and functional information, and tissue characteristics as disclosed in International PCT Application No. PCT/IB2019/000963 (Published as WO 2020/044117) to Hennersperger et al., U.S. Application Publication No. US 2022-0287679A1 to Hennersperger et al., and U.S. Pat. No. 11,382,599 to Hennersperger et al., the contents of each which are incorporated by reference herein.

The systems and methods of the invention exploit properties of the ultrasound data generated by imaging systems, and sample only when the received data has changed sufficiently, for example, according to a previously defined threshold classified as an “event.” The threshold may have been determined, for example, based on the clinical relevance of the structures being imaged and the characteristics of the received data. For example, the threshold may be defined to avoid sampling the blood pool, which may not be clinically relevant, at a high data rate. Aspects of the invention provide a system for image sampling comprising a hardware processor coupled to non-transitory computer-readable memory. The computer-readable memory contains instructions executable by the processor to cause the processor to receive data from an imaging device; and run an event-driven sampling algorithm. As discussed in more detail below, the algorithm causes the processor to analyze the received data to identify one or more characteristics associated with the received data and further detect the occurrence of one or more events. The one or more events may include a change in the one or more characteristics as compared to a defined threshold for the one or more characteristic. The algorithm causes the processor to then tune a sampling rate of the imaging device based on a detected occurrence of the one or more events.

### Ultrasound Imaging System

By way of overview, and as is generally understood, ultrasound imaging (sonography) uses high-frequency sound waves to view inside the body. Because ultrasound images are captured in real-time, these images can also show movement of the body's internal organs as well as fluid flow (e.g., blood flowing through blood vessels). In an ultrasound exam, the imaging device, (i.e. the transducer, probe, or transducer probe) is placed directly on the skin or inside a body opening (e.g. endovascular ultrasound, intravascular ultrasound, intracardiac echocardiography).

Systems of the invention are configured to receive data from an imaging device, such as three-dimensional (3D) ultrasound image data.

In some embodiments, the invention provides for event-driven sampling that allows for three-dimensional visualization and tissue characterization in minimally invasive procedures in the vasculature. Accordingly, ultrafast ultrasound imaging techniques, such as planewave or diverging wave imaging, may be required to enable imaging within the constraints of the application, particularly for intravascular and/or intracardiac tissue assessment and analysis. Systems and methods of the invention allow for the direct utilization of all native ultrafast imaging techniques.

For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data the overall image may be reconstructed at once in parallel by dynamically beamforming the received RF data for each target position.

Ultrafast ultrasound methods offer imaging at thousands of frames per second limited only by the physical propagation speed of sound waves in tissue, and enable ultrasensitive blood-flow tracking, shear-wave imaging, super-resolution imaging, and other applications. For example, achieving optimal spatial resolution while enabling artifact-free imaging of dynamic cardiac structures requires a careful balance between spatial sampling and volumetric update rate which can only be achieved using ultrafast imaging techniques. Thus, the 3D ultrasound image data received by systems of the invention may be real-time 3D ultrasound data. For example, the data may be full circumferential, three-dimensional (3D) image data.

Systems of the invention may be operably coupled to a catheter-based ultrasound imaging device operably coupled to a console and configured to transmit ultrasound pulses to, and receive echoes of the ultrasound pulses from, intravascular and/or intracardiac tissue.

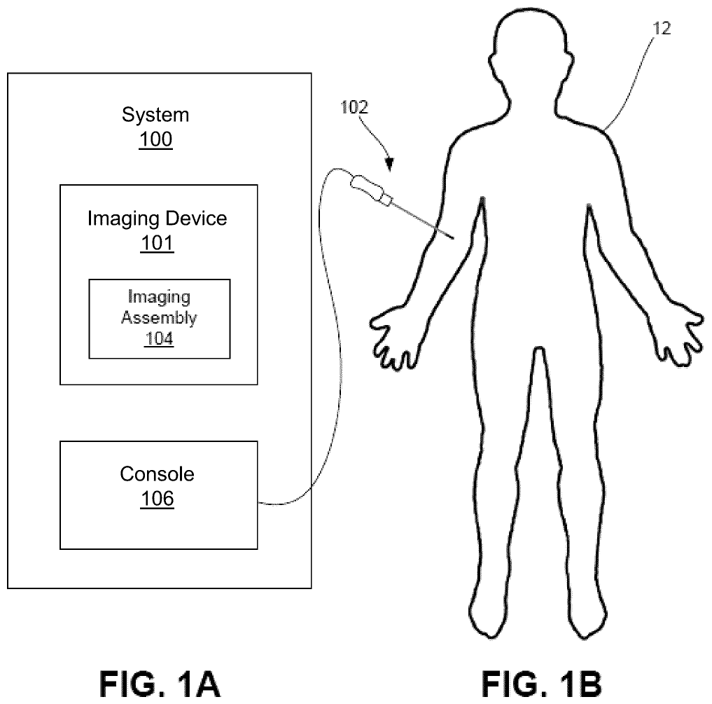

FIG. 1A and FIG. 1B are diagrammatic illustrations of an exemplary system 100 for providing visualization and characterization of tissue and/or vasculature within a patient 12 with which systems and methods of the invention may be used. The system 100 may include an imaging device 101 equipped with an imaging assembly 104 and a console 106 to which the imaging device 101 is to be connected. The imaging device may be an imaging catheter 102. Accordingly, systems and methods for event-driven sampling disclosed herein may use a 4D Intracardiac echocardiogram (ICE) system that captures the anatomy of interest. ICE is a catheter-based form of echocardiography that gathers images from within the heart, rather than by gathering images of the heart by sending sound waves through the chest wall. Specific embodiments of the systems and methods of the invention may be used with a four-dimensional (4D) catheter-based ultrasound imaging device.

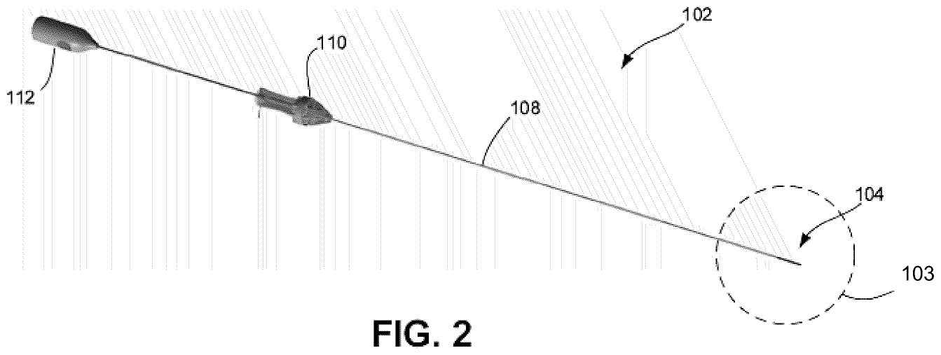

FIG. 2 is a perspective view of an imaging catheter 102 with which systems of the invention may be coupled. The catheter 102 may include a catheter body 108, including proximal and distal portions. The imaging assembly 104 may be provided at the distal portion, for example, generally defining a distal end of an imaging catheter. A handle 110 may be operably associated with the catheter body 108 and allow for an operator (i.e., surgeon or other medical professional) to manipulate and advance the imaging assembly 104 and the catheter body 108 to a desired target site within the patient's vasculature. The handle 110 may include user-operable inputs for controlling various features and functions of the imaging assembly 104. An interface member 112 may be provided at a distal portion of the catheter body 108. The interface member 112 generally provides a connection between the imaging catheter 102, including the imaging assembly 104 and handle 110, and the console 106 for transmission of signals therebetween. The connection may include at least one of a hardwired and wireless connection, for example.

In certain embodiments of the systems, the console may be configured to receive at least full circumferential, 3D image data from the ultrasound imaging device in real, or near-real time. Thus, the console may be configured to reconstruct multiple images in real, or near-real time, based, at least in part, on user input and/or predefined protocols.

The console may be operably coupled to the imaging device and may generally control operation of the transducer probe i.e., transmission of sound waves from the probe. The console may generally include one or more processors (e.g., a central processing unit (CPU), a graphics processing unit (GPU), or both) and storage, such as main memory, static memory, or a combination of both, which communicate with each other via a bus or the like. The memory according to embodiments of the invention may include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

### Systems for Event-Driven Ultrasound Imaging

As noted above, systems of the invention provide for event-driven ultrasound sampling.

Aspects of the invention include a system for image sample comprising a hardware processor coupled to non-transitory, computer-readable memory containing instructions executable by the processor to cause the processor to receive data from an imaging device; and run an event-driven sampling algorithm.

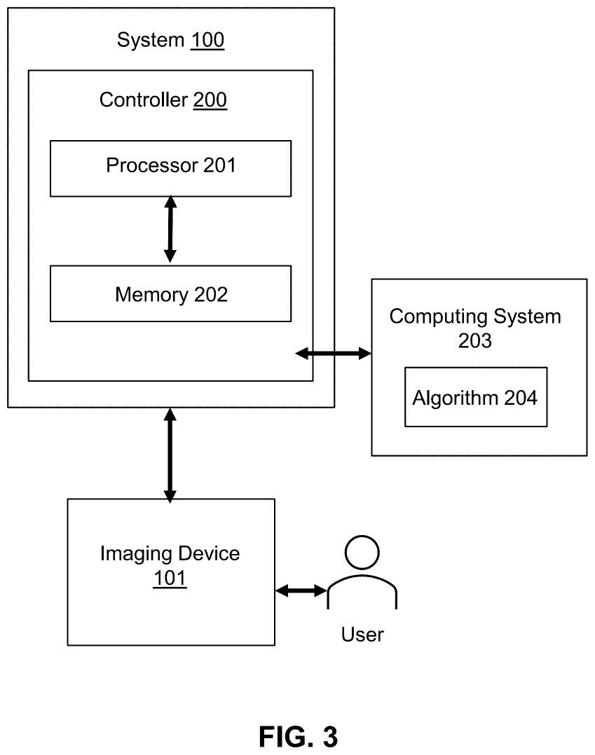

FIG. 3 illustrates one embodiment of systems 100 of the invention. For example, the system 100 may include a controller 200 comprising a hardware processor 201 coupled to non-transitory computer-readable memory 202 containing instructions executable by the processor 201. The controller may be in active communication with a computing system 203 configured to communicate across a network. The controller may be operational as software, on a microprocessor, and/or may be embedded as part of a custom ASIC/Chip.

The computing system 203 or computing device may include one or more processors and memory, as well as an input/output mechanism (i.e., a keyboard, knobs, scroll wheels, or the like) with which an operator can interact so as to operate the machine and/or device, including making adjustments to the transmission characteristics of the ultrasound probe and/or device, saving images, and performing other tasks described herein, including selection of specific regions of interest for subsequent reconstruction into 2D and/or 3D images.

During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. As disclosed herein, the system may include a console 106 to further provide control over an imaging assembly, including control over the emission of ultrasound pulses therefrom (intensity, frequency, duration, etc.) as well as control over the movement of the ultrasound transducer unit. Control over the imaging assembly may be by the controller, processor, and/or the computing system. Thus, the system may be in communication with the imaging device 101 to receive 3D ultrasound image data from the imaging device 101.

The computing system 203 may include a computer program comprising an event-driven algorithm 204, such that the computer-readable memory 202, containing instructions executable by the processor 201, causes the processor to receive data from the imaging device 101 and run one or more event-driven algorithms 204. The event-driven algorithm 204 may be part of a computer program executable by the computing system 203 and in communication with the console of the system 100. The system may also include one or more algorithms for dynamically reconstructing multiple images from the 3D image data to provide a 3D visualization of the anatomical region of interest and targeted tissue site.

The event-driven algorithm causes the processor to analyze the received data to identify one or more characteristics associated with the received data and further detect the occurrence of one or more events. The one or more events include a change in the one or more characteristics as compared to a defined threshold for the one or more characteristic. The event-driven algorithm also causes the processor to tune a sampling rate of the imaging device based on a detected occurrence of the one or more events.

The event-driven algorithm may be one or more of a differential algorithm, a difference threshold algorithm, a moving threshold algorithm, a logarithmic algorithm, a triggered burst algorithm, and an in-phase quadrature detection algorithm.

For example, for the differential algorithm, the output voltage may be encoded at a lower bit depth than full sampling (16-bit). The resulting signal may then sampled whenever the output voltage changes.

For example, for the difference threshold algorithm, the waveform may be sampled with the output y(t) changes relative to the previous value y(t-1) by a parameter ±Δ.

For example, for the moving threshold algorithm, the waveform may be sampled with the output voltage changes relative to a previously stored threshold value by a parameter ±Δ. The threshold value may be updated and stored whenever sampling is triggered.

As an example, for the logarithmic algorithm, the waveform may be sampled whenever the output voltage crossed any number of logarithmically spaced fixed voltage levels.

In another example, for the triggered burst algorithm, the waveform may be sampled at full sampling rate for a set time T any time the output voltage y(t) changes relative to the previous value y(t-1) by a parameter ±Δ.

For example, for the in-Phase quadratic detection algorithm, only the amplitude and phase of the carrier wave (extracted via IQ demodulation) may be sampled, with the amplitude signal sampled according to the “moving threshold” algorithm and the phase sampled at the same time as the amplitude and encoded at a lower bit rate than 16 bits.

Notably, the event-driven ultrasound sampling using the algorithms described herein may be implemented in silicon, such that raw datarates can be reduced from the analog frontend of the system and also during further processing. Thus, the systems and methods of the invention provide advantages by reducing datarates on communication links (e.g. when transferring data through small cable assemblies or limited wire count in catheters). The systems and methods of the invention also provide advantages by reducing total data size, (e.g. for storing information persistently, where data volume swiftly becomes prohibitive).

In some embodiments, the defined threshold is determined based, at least in part, on a clinical relevance of a structure to be imaged and/or one or more characteristics of the received data. For example, the blood pool may not be clinically relevant. Therefore the threshold may be defined to avoid sampling the blood pool at a high data rate.

The received data may comprise digitized voltage signals or analog voltage signals. In some embodiments, the processor is configured to access raw digitized voltage signals, and tune one or more sampling parameters to minimize the data transmission rate and to maximize an image quality of a clinically relevant image.

As disclosed herein, the characteristics of the data may be any characteristic associated with the received data, and the defined threshold may be any threshold defined for the characteristic. For example, in some embodiments, the characteristic may be a digitized voltage, and the defined threshold may be the digitized voltage encoded at a lower bit depth than a full sampling bit depth. Bit depth, as used herein is the number of bits per pixel. In some embodiments, the defined threshold may be a change in output voltage by a defined parameter relative to a previous output voltage. For example, the image sampling by the imaging device may be triggered at a full sampling rate for a set time for each occurrence of an event associated with the output voltage changing by the defined parameter relative to the previous output voltage. In some embodiments, the defined threshold may be a change in output voltage by a set parameter in a previously stored threshold value, such that the threshold value is updated and stored when image sampling by the imaging device is triggered. In some embodiments, the defined threshold is one or more logarithmically spaced voltage levels.

As described in the examples below, the defined threshold may be a change in an amplitude signal by a set parameter in a previously stored threshold amplitude signal, such that only amplitude and phase of a carrier wave are sampled with the phase sampled concurrently with the amplitude and encoded at a lower bit rate than 16 bits. The amplitude and the phase of the carrier wave may be extracted via I/Q demodulation. As known to persons skilled in the art, I/Q demodulation mixes an in-phase and quadrature-phase sinusoid with the input signal, causing signal content of that frequency to be accentuated and all other content to be reduced.

In some embodiments, the imaging device comprises a transducer comprising an array of individual imaging elements. For example, the transducer comprises a micro-electromechanical systems (MEMS)-based micromachined ultrasonic transducer configured as a two-dimensional (2D) array structure.

In the present invention, the transducer may be any type for transmitting and receiving acoustic waves. For example, the transducer may include one-or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices do not require bias for achieving imaging sensitivity.

The transducer may be a micro-electromechanical systems (MEMS)-based capacitive micromachined ultrasonic transducer (CMUT) configured as a two-dimensional (2D) array structure. In non-limiting examples, the 2D array may be a flexible structure. The cylindrical imaging array may consist of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13(10) 3873-3882, incorporated herein by reference.

Additionally and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers may be made of electrostrictive materials such as an electrostrictive polymer, or any material that can be activated using bias voltage to achieve imaging sensitivity.

In some embodiments, the imaging elements are acoustic sensors activated by the processor to transmit and/or receive a plurality of incident acoustic wave signals as wave data. The wave data may be, for example at least one of plane wave data and diverging wave data associated with one or more plane wave transmit-receive cycles carried out by the imaging elements. The active elements used for transmit and receive may comprise all elements in both transmit and receive, an identical subset of elements used for transit and receive, or a different subset (or full set) of elements used for transmit and receive of wave data. The wave data may be full circumferential, three-dimensional (3D) image data.

In some embodiments, the imaging device may be a catheter-based ultrasound imaging device configured to transmit ultrasound pulses to, and receive echoes of the ultrasound pulses from, intravascular and/or intracardiac tissue. For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data, the overall image may be reconstructed at once in parallel by dynamically beamforming the received RF data for each target position.

In some embodiments, the imaging device may be a minimally invasive implantable device. For example, the processor may be embedded as part of an application-specific integrated circuit. The system may be embedded as part of a custom ASIC/chip to control the event-driven sampling scheme, such that the acoustic sound waves deliver power and telemetry capabilities to implanted devices for remote sensing of the physiological environment in soft biological tissues, as an alternative to inductive (near field) and radio-frequency (RF) links. The processor may be part of integrated components on a flexible membrane chip wherein the core component is a wireless ultrasonic energy converter that couples ultrasonic mechanical vibrations with a triboelectric generator to convert mechanical to electrical energy. Accordingly, the event-driven scheme may include tuning the sampling rate such that the overall power consumption and heat dissipation of the device is reduced.

In some embodiments, tuning of the sampling rate results in reduction of an average sampling rate of received data from the imaging device.

### Methods for Event-Driven Ultrasound Sampling

Aspects of the invention provide for methods for image sampling.

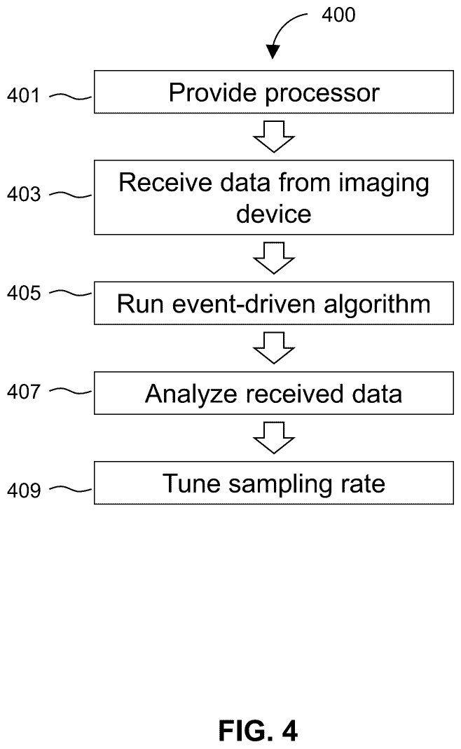

FIG. 4 is a block diagram illustrating a method for event-driven ultrasound according to one embodiment of the invention.

The method 400 includes the steps providing 401 a hardware processor coupled to non-transitory, computer-readable memory containing instructions executable by the processor to cause the processor to receive 403 data from an imaging device; run 405 an event-driven sampling algorithm causing the processor to; analyze 407 the received data to identify one or more characteristics associated with the received data and further detect the occurrence of one or more events, wherein the one or more events comprises a change in the one or more characteristics as compared to a defined threshold for the one or more characteristic; and tune 409 a sampling rate of the imaging device based on a detected occurrence of the one or more events.

As disclosed herein, the controller may include a hardware processor coupled to non-transitory computer-readable memory containing instructions executable by the processor. The controller may be in active communication with a computing system configured to communicate across a network. The controller may be operational as software, on a microprocessor, and/or may be embedded as part of a custom ASIC/Chip. The computing system or computing device may include one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for subsequent reconstruction into 2D and/or 3D images.

During operation, the CPU and/or GPU may control the transmission and receipt of electrical currents, subsequently resulting in the emission and receipt of sound waves from the probe. The CPU and/or GPU may also analyze electrical pulses that the probe makes in response to reflected waves coming back and then may converts this data into images (i.e., ultrasound images) that can then be viewed on a display, which may be an integrated monitor. Such images may also be stored in memory and/or printed via a printer. As disclosed herein, the system may include a console (not shown) to further provide control over an imaging assembly, including control over the emission of ultrasound pulses therefrom (intensity, frequency, duration, etc.) as well as control over the movement of the ultrasound transducer unit. Control over the imaging assembly may be by the controller, processor, and/or the computing system. Thus, the system may be in communication with the imaging device to receive 3D ultrasound image data from the imaging device.

The computing system may include a computer program comprising an event-driven algorithm, such that the computer-readable memory, containing instructions executable by the processor, causes the processor to receive data from the imaging device and run one or more event-driven algorithms. The event-driven algorithm may be part of a computer program executable by the computing system and in communication with the console of the system. The system may also include one or more algorithms for dynamically reconstructing multiple images from the 3D image data to provide a 3D visualization of the anatomical region of interest and targeted tissue site.

In methods of the invention, the event-driven algorithm causes the processor to analyze the received data to identify one or more characteristics associated with the received data and further detect the occurrence of one or more events. The one or more events include a change in the one or more characteristics as compared to a defined threshold for the one or more characteristic. The event-driven algorithm also causes the processor to tune a sampling rate of the imaging device based on a detected occurrence of the one or more events.

As disclosed herein, the event-driven algorithm of the methods may be one or more of a differential algorithm, a difference threshold algorithm, a moving threshold algorithm, a logarithmic algorithm, a triggered burst algorithm, and an in-phase quadrature detection algorithm.

For example, for the differential algorithm, the output voltage may be encoded at a lower bit depth than full sampling (16-bit). The resulting signal may then sampled whenever the output voltage changes.

For example, for the difference threshold algorithm, the waveform may be sampled with the output y(t) changes relative to the previous value y(t-1) by a parameter ±Δ.

In another example, for the moving threshold algorithm, the waveform may be sampled with the output voltage changes relative to a previously stored threshold value by a parameter ±Δ. The threshold value may be updated and stored whenever sampling is triggered. For example, for the logarithmic algorithm, the waveform may be sampled whenever the output voltage crosse any number of logarithmically spaced fixed voltage levels.

Further, for the triggered burst algorithm, the waveform may be sampled at full sampling rate for a set time T any time the output voltage y(t) changes relative to the previous value y(t-1) by a parameter ±Δ.

In another example, for the in-Phase quadratic detection algorithm, only the amplitude and phase of the carrier wave (extracted via IQ demodulation) may be sampled, with the amplitude signal sampled according to the “moving threshold” algorithm and the phase sampled at the same time as the amplitude and encoded at a lower bit rate than 16 bits.

Notably, the event-driven ultrasound sampling using the algorithms described herein may be implemented in silicon, such that raw datarates can be reduced from the analog frontend of the system and also during further processing. Thus, the systems and methods of the invention provide advantages by reducing datarates on communication links (e.g. when transferring data through small cable assemblies or limited wire count in catheters). The systems and methods of the invention also provide advantages by reducing total data size, (e.g. for storing information persistently, where data volume swiftly becomes prohibitive).

In some embodiments of the methods, the defined threshold is determined based, at least in part, on a clinical relevance of a structure to be imaged and/or one or more characteristics of the received data. For example, the blood pool may not be clinically relevant. Therefore the threshold may be defined to avoid sampling the blood pool at a high data rate.

The received data may comprise digitized voltage signals or analog voltage signals. In some embodiments, the processor is configured to access raw digitized voltage signals, and tune one or more sampling parameters to minimize the data transmission rate and to maximize an image quality of a clinically relevant image.

As disclosed herein, the characteristics of the data may be any characteristic associated with the received data, and the defined threshold may be any threshold defined for the characteristic. For example, in some embodiments of the methods, the characteristic may be a digitized voltage, and the defined threshold may be the digitized voltage encoded at a lower bit depth than a full sampling bit depth. Bit depth, as used herein is the number of bits per pixel. In some embodiments, the defined threshold may be a change in output voltage by a defined parameter relative to a previous output voltage. For example, the image sampling by the imaging device may be triggered at a full sampling rate for a set time for each occurrence of an event associated with the output voltage changing by the defined parameter relative to the previous output voltage. In some embodiments, the defined threshold may be a change in output voltage by a set parameter in a previously stored threshold value, such that the threshold value is updated and stored when image sampling by the imaging device is triggered. In some embodiments, the defined threshold is one or more logarithmically spaced voltage levels.

As described in the examples below, the defined threshold may be a change in an amplitude signal by a set parameter in a previously stored threshold amplitude signal, such that only amplitude and phase of a carrier wave are sampled with the phase sampled concurrently with the amplitude and encoded at a lower bit rate than 16 bits. The amplitude and the phase of the carrier wave may be extracted via I/Q demodulation. As known to persons skilled in the art, I/Q demodulation mixes an in-phase and quadrature-phase sinusoid with the input signal, causing signal content of that frequency to be accentuated and all other content to be reduced.

In some embodiments of the methods, the imaging device comprises a transducer comprising an array of individual imaging elements. For example, the transducer may include a micro-electromechanical systems (MEMS)-based micromachined ultrasonic transducer configured as a two-dimensional (2D) array structure.

In the present methods of the invention, the transducer may be any type for transmitting and receiving acoustic waves. For example, the transducer may include one-or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

CMUT devices offer excellent bandwidth and acoustic impedance characteristics, which makes these transducers preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an ASIC, facilitates both transmission and reception modes of the device. In a reception mode, changes in the membrane position cause changes in electrical capacitance, which can be registered electronically while in a transmission mode, applying an electrical signal causes vibration of the membrane.

Piezoelectric micro-machined ultrasound transducers (PMUT) are based on the flexural motion of a thin membrane coupled with a thin piezoelectric film, such as PVDF. This is in comparison to bulk piezoelectric transducers which use the thickness-mode motion of a plate of piezoelectric ceramic such as PZT or single-crystal PMN-PT. In comparison with bulk piezoelectric ultrasound transducers, PMUT devices offer advantages such as increased bandwidth, flexible geometries, natural acoustic impedance matched with water, reduced voltage requirements, mixing of different resonant frequencies and potential for integration with supporting electronic circuits especially for miniaturized high frequency applications. Current PMUT devices do not require bias for achieving imaging sensitivity.

The transducer may be a micro-electromechanical systems (MEMS)-based capacitive micromachined ultrasonic transducer (CMUT) configured as a two-dimensional (2D) array structure. In non-limiting examples, the 2D array may be a flexible structure. The cylindrical imaging array may consist of a flexible 2D-array structure in a CMUT design. Flexible MEMS-based arrays may be implemented, for example, by wafer thinning (CMUT/PMUT), or by using specific approaches such as combining rigid imaging cells with flexible interconnects as described in Mimoun, 2013, A generic platform for the fabrication and assembly of flexible sensors for minimally invasive instruments, IEEE Sensors J 13(10) 3873-3882, incorporated herein by reference.

Additionally and/or alternatively, the transducer may be made of an electrostrictive material configured as a two-dimensional (2D) array structure. Electrostriction is a property of all dielectric materials and consists of a mechanical displacement as a response to an electronic field, such as material compression in the regions of high electric field strength. In electrostriction, an electric field applied to the material generates the deformation of the material (direct effect), and a mechanics stress applied to the material changes the material polarization (inverse effect). Transducers may be made of electrostrictive materials such as an electrostrictive polymer, or any material that can be activated using bias voltage to achieve imaging sensitivity.

In some embodiments of the methods, the imaging elements are acoustic sensors activated by the processor to transmit and/or receive a plurality of incident acoustic wave signals as wave data. The wave data may be, for example at least one of plane wave data and diverging wave data associated with one or more plane wave transmit-receive cycles carried out by the imaging elements. The active elements used for transmit and receive may comprise all elements in both transmit and receive, an identical subset of elements used for transit and receive, or a different subset (or full set) of elements used for transmit and receive of wave data. The wave data may be full circumferential, three-dimensional (3D) image data.

In some embodiments of the method, the imaging device may be a catheter-based ultrasound imaging device configured to transmit ultrasound pulses to, and receive echoes of the ultrasound pulses from, intravascular and/or intracardiac tissue. For example, for intracardiac imaging, planewave imaging may refer to an ultrasound imaging modality where, through a flat transmit of all transducer elements (at different angles) from the angular imaging aperture, a plane wave front may traverse the tissue and may be partially scattered back to the transducer. From the received radio frequency (RF) (i.e. channel) data, the overall image may be reconstructed at once in parallel by dynamically beamforming the received RF data for each target position.

In some embodiments of the method, the imaging device may be a minimally invasive implantable device. For example, the processor may be embedded as part of an application-specific integrated circuit. The system may be embedded as part of a custom ASIC/chip to control the event-driven sampling scheme, such that the acoustic sound waves deliver power and telemetry capabilities to implanted devices for remote sensing of the physiological environment in soft biological tissues, as an alternative to inductive (near field) and radio-frequency (RF) links. The controller may be part of integrated components on a flexible membrane chip wherein the core component is a wireless ultrasonic energy converter that couples ultrasonic mechanical vibrations with a triboelectric generator to convert mechanical to electrical energy. Accordingly, the event-driven scheme may include tuning the sampling rate such that the overall power consumption and heat dissipation of the device is reduced.

In some embodiments of the method, tuning of the sampling rate results in reduction of an average sampling rate of received data from the imaging device.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions, or instruction sets and/or data that are hard-coded (e.g., nonvolatile) in memory devices.

“Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions.

Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### EXAMPLE

Development of the event-driven sampling architecture used the protocol disclosed herein and tested using the below algorithms:
DIFFERENTIAL: reduce bit depth of signal according to bit_level and sample only when signal changes. Output bit level is computed as 16-bit_level so higher bit_level corresponds to reduced bit depth of sampled signal.
DIFFERENCE_THRESHOLD: sample only when signal changes by an amount delta relative to the most recently measured value.
MOVING_THRESHOLD: sample only when signal changes by an amount delta relative to a threshold that is updated when sampling is triggered.
TRIGGERED_BURST: mix of normal and event-driven sampling, when signal changes by a set amount delta, a finite number of samples (parameter burst_length) is collected as normal.
QUADRATURE_PHASE: the amplitude and phase of the modulated carrier wave at frequency f_c is extracted, and the amplitude is sampled using:
   The amplitude A(t) and phase ϕ(1) are computed using the in-phase and quadrature components of the channel data, where the channel data for a single channel and event takes the form y(t)=A(t) sin(2πf_{c}t+Ø(t))+C(t)
   The IQ signal is computed by multiplying the rectified channel data (i.e. channel data with the offset C (t) removed) by a carrier wave and quadrature shifted carrier wave: IQ=y_{DC}(t)×e^{i2πf}^{_{c}}^{t}=y_{DC}(t)[cos(2πf_{c}t)+i sin(2πf_{c}t)]
   The resulting IQ signal has the form (using trigonometric product-to-sum identities):

$$IQ = \frac{A ( t )}{2} [ sin ( 2 π 2 {f}_{c} t + ϕ ( t ) ) + sin ( ϕ ( t ) ) + i ( cos 2 π2 {f}_{c} t + ϕ ( t ) ) + cos ( ϕ ( t ) ) ) ]$$

The terms occurring at 2f_{c} can be removed using a low pass filter, and the filtered IQ signal has the form:

$$= \frac{A ( t )}{2} ( ( sin ϕ ( t ) ) + i cos ϕ ( t ) )$$

$$A ( t ) = 2 \times \vert \vert$$

$$ϕ ( t ) = {sin}^{- 1} ( \frac{Re ( )}{\vert \vert} ) or {cos}^{- 1} ( \frac{Im ( )}{\vert \vert} )$$

The amplitude is then sampled according to the MOVING_THRESHOLD algorithm and the phase is sampled using the DIFFERENTIAL algorithm
The modified channel data is reconstructed as LOGARITHMIC_THRESHOLD: fixed level crossing values at logarithmically increasing steps
   Algorithm training and Testing

OPSFs are modified and beamformed as normal. The quality of the resulting beamformed images (orbital slices or compounded volumes) was compared to the unmodified images using mean squared error (MSE). Other similarity metrics were structural similarity (SSIM) and peak signal-to-noise (PSNR).

### Synthetic Data

Data generated was modified and the resulting beamformed images compared OPSF analyzed:
Shared Data\SimulationStudies\Event-Driven_Analysis\sp2sp\sp2sp\train.opsf
Snapshots: 00 and 01
Frames: all (00000-00004 for both snapshots)

### Pre-Clinical Data

Max identified some representative datasets from the preclinical at IMMR in July 2022

### OPSFs Analyzed

2022-07-28_IMMR\002_VeraStudy_c56ac7a0-c940-4ba3-aaef-d13e386d0a0c
Snapshot 01: RA home view anatomical rotational imaging
Frames: 0001-00141 in steps of 10 frames

2022-07-28_IMMR\004_VeraStudy_5390e68c-4a62-4600-8b35-953c915c5877.opsf
Snapshot 01: Pullback from SVC to IVC
Frames: 0001-00401 in steps of 50 frames

### Reduced Sampling Rate

Results from QUADRATURE_PHASE algorithm suggested that sampling the signal at a lower rate, since the envelope varies at a much lower frequency than the carrier. Sampling is in excess of the Nyquist rate (example carrier frequency is 6.25 MHz, sampling rate is 32 MHz) so likely can reduce the sampling rate without significant loss of quality.

### Results

### Synthetic Data

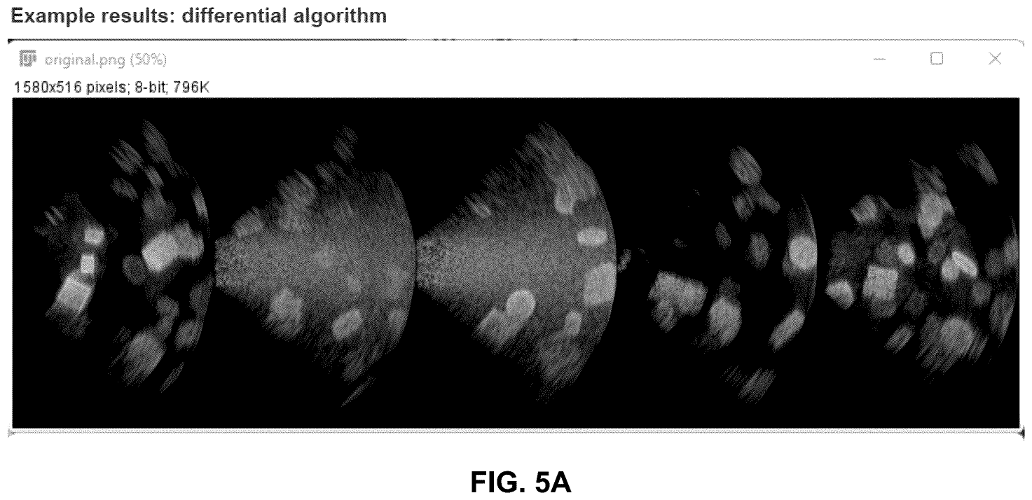

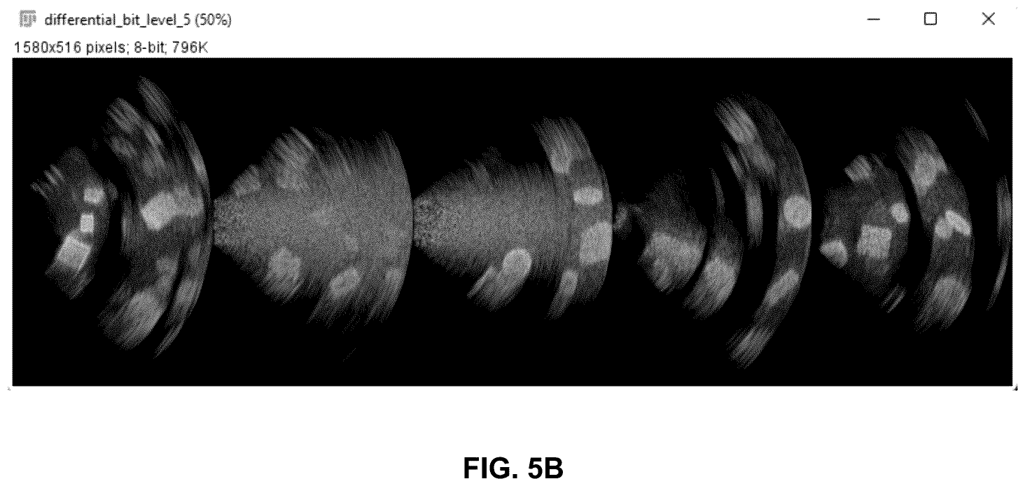

FIGS. 5A and 5B show example results comparing the original image and the image created using the differential algorithm. FIG. 5A illustrates the original png file at 50% 1580×516 pixels; 8-bit, 796K. FIG. 5B illustrates the image from the differential algorithm differential_bit_level_5 at 50%, 1580×516 pixels; 8-bit; 796K.

### Summary of Algorithm Performance—Synthetic Data

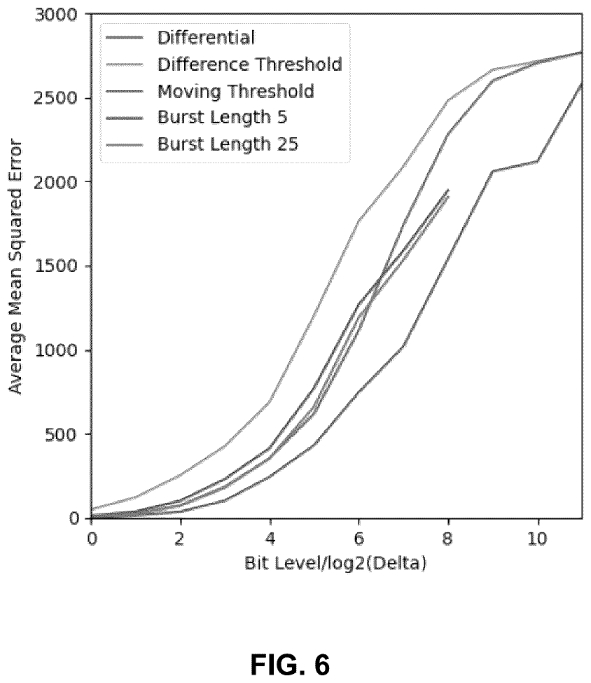

FIG. 6 is a graph illustrating the average mean squared error for the differential algorithm, the difference threshold algorithm, the moving threshold algorithm and the triggered burst algorithm (using a burst length of 5 and 25), per bit level/log2 (Delta).

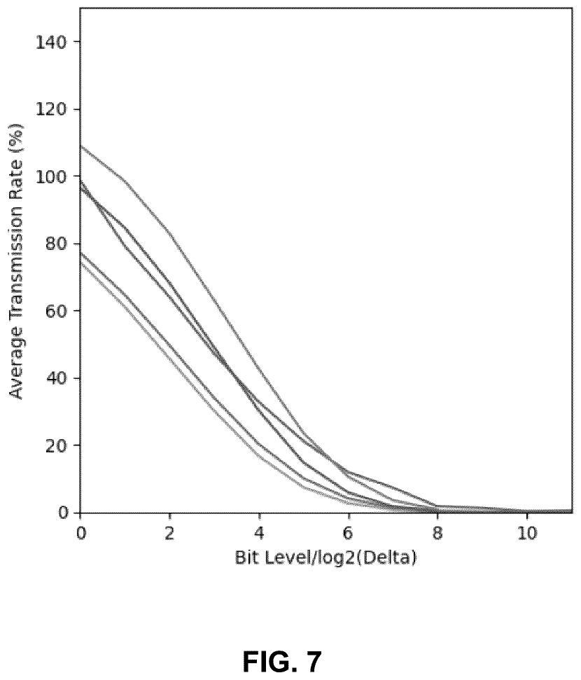

FIG. 7 is a graph illustrating the average transmission rate (%) for the differential algorithm, the difference threshold algorithm, the moving threshold algorithm and the triggered burst algorithm (using a burst length of 5 and 25), per bit level/log2 (Delta).

### Summary of Algorithm Performance—Anatomical Imaging in RA

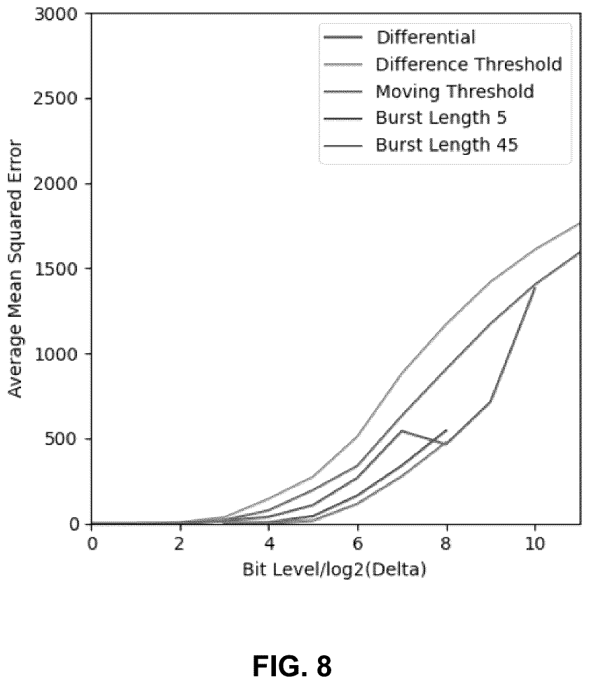

FIG. 8 is a graph illustrating the average mean squared error (Pre-Clinical Data (2022-07-28_IMMR)) for the differential algorithm, the difference threshold algorithm, the moving threshold algorithm and the triggered burst algorithm (using a burst length of 5 and 45), per bit level/log2 (Delta).

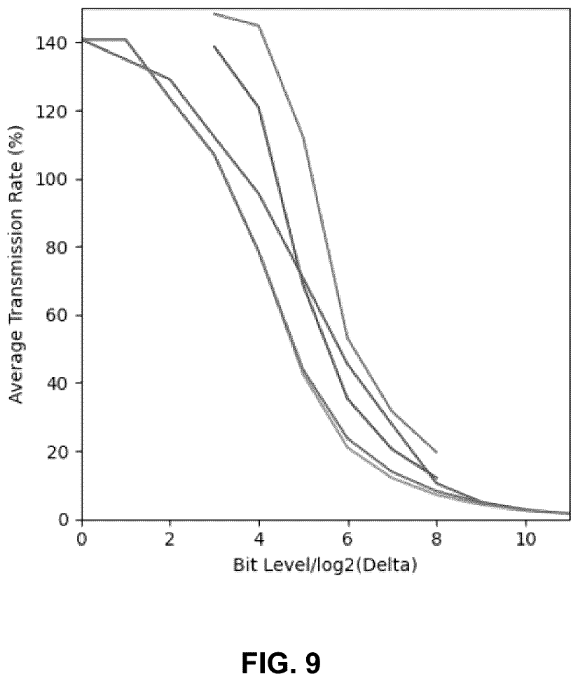

FIG. 9 is a graph illustrating the average transmission rate (%) (Pre-Clinical Data (2022-07-28_IMMR)) for the differential algorithm, the difference threshold algorithm, the moving threshold algorithm and the triggered burst algorithm (using a burst length of 5 and 45), per bit level/log2 (Delta).

### Summary of Algorithm Performance—Pullback Sequence

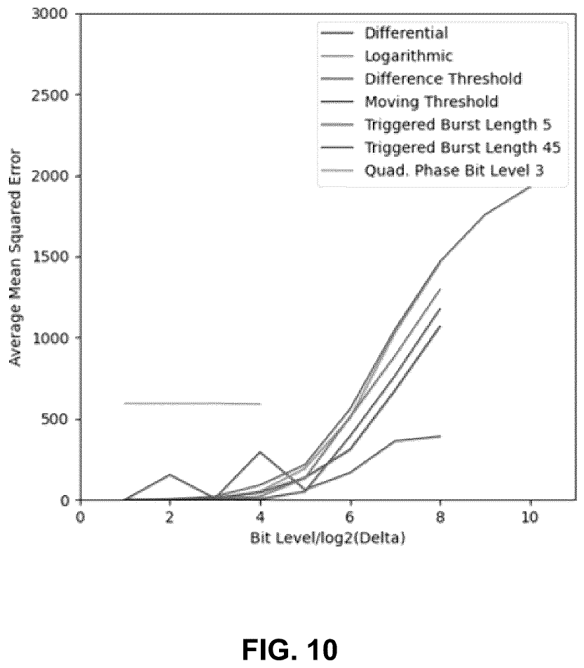

FIG. 10 is a graph illustrating the average mean squared error for the differential algorithm, the logarithmic algorithm, the difference threshold algorithm, the moving threshold algorithm, the triggered burst algorithm (burst length 5), the triggered burst algorithm (burst length 45) and the quadratic phase algorithm (bit level 3), per bit level/log2 (Delta).

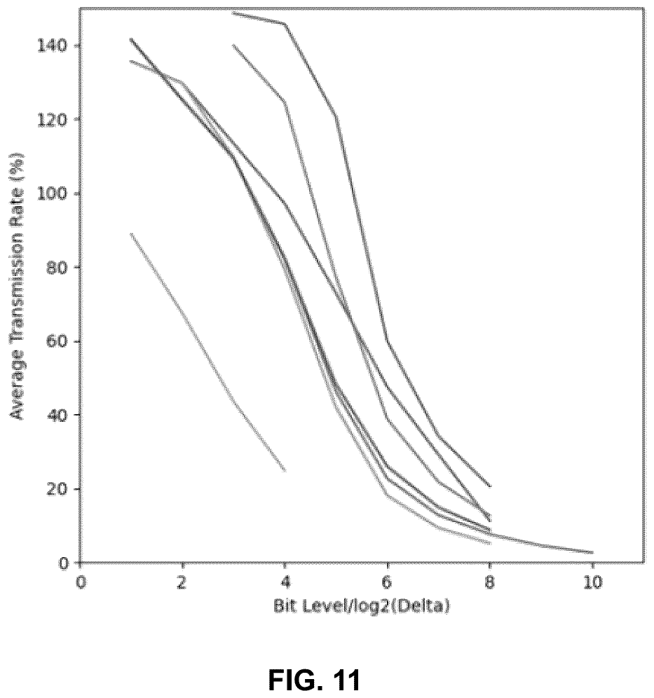

FIG. 11 is a graph illustrating the average transmission rate (%) for the differential algorithm, the logarithmic algorithm, the difference threshold algorithm, the moving threshold algorithm, the triggered burst algorithm (burst length 5), the triggered burst algorithm (burst length 45) and the quadratic phase algorithm (bit level 3), per bit level/log2 (Delta).

### Data Transmission Histograms

The histograms for each algorithm represents a fixed parameter level of 6 for the pullback sequence using the preclinical data.

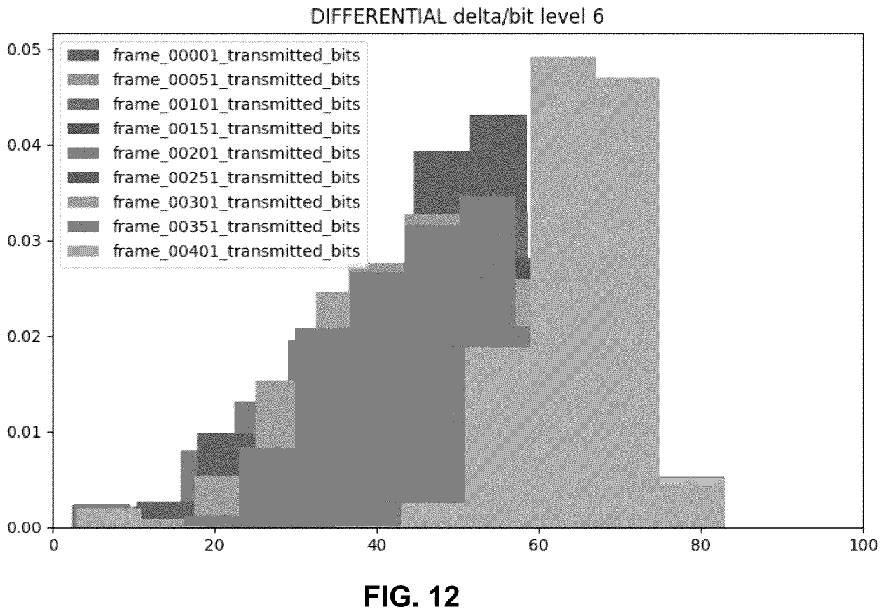

FIG. 12 illustrates the data transmission histogram for the DIFFERENTIAL algorithm with delta/bit level 6.

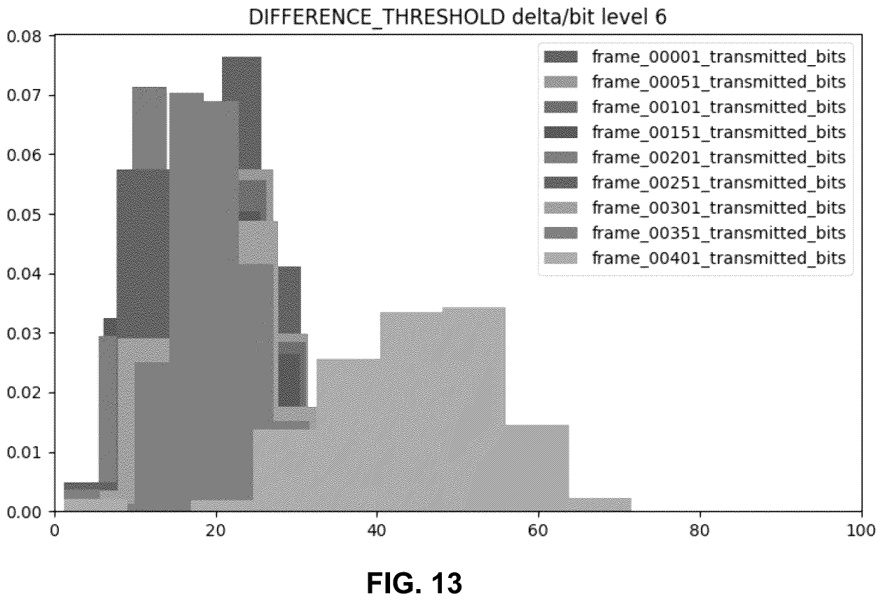

FIG. 13 illustrates the data transmission histogram for the DIFFERENCE_THRESHOLD algorithm with delta/bit level 6.

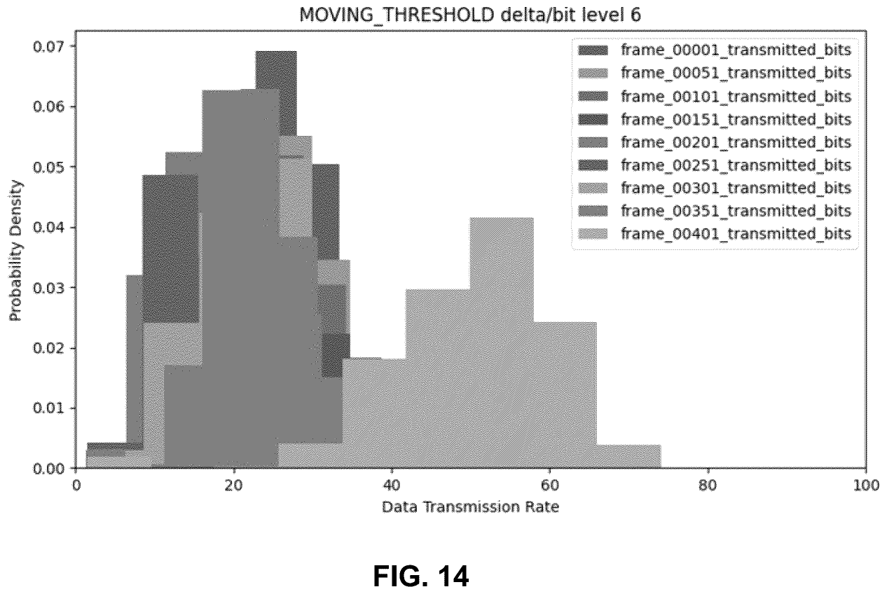

FIG. 14 illustrates the data transmission histogram for the MOVING_THRESHOLD algorithm with delta/bit level 6.

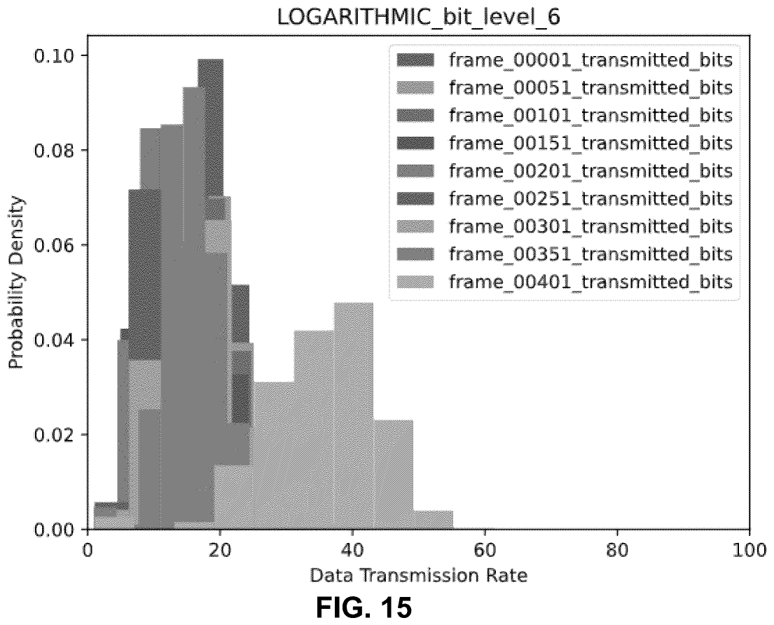

FIG. 15 illustrates the data transmission histogram for the LOGARITHMIC_bit_level_6 algorithm with delta/bit level 6.

### Reduced Sampling Rate

Evaluated DIFFERENTIAL, MOVING_THRESHOLD, and LOGARITHMIC ALGORITHMS. Image quality appeared poor for QUADRATURE_PHASE algorithm due to high-pass filtering of second harmonics.

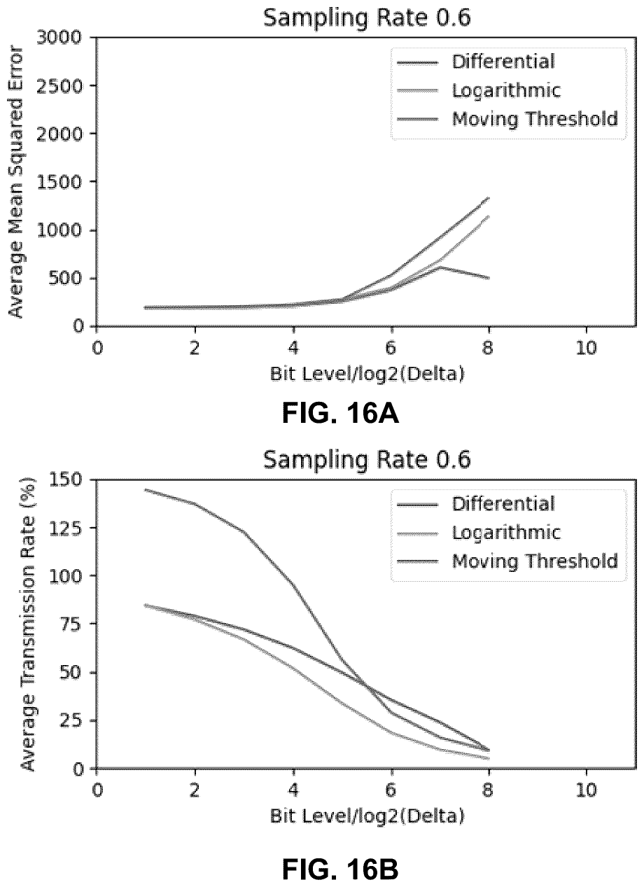

FIGS. 16A and 16B illustrate the average mean squared error and average transmission rate (%) versus Bit Level/log2 (Delta) using a sampling rate of 0.6 for the Differential, Logarithmic, and Moving Threshold algorithms.

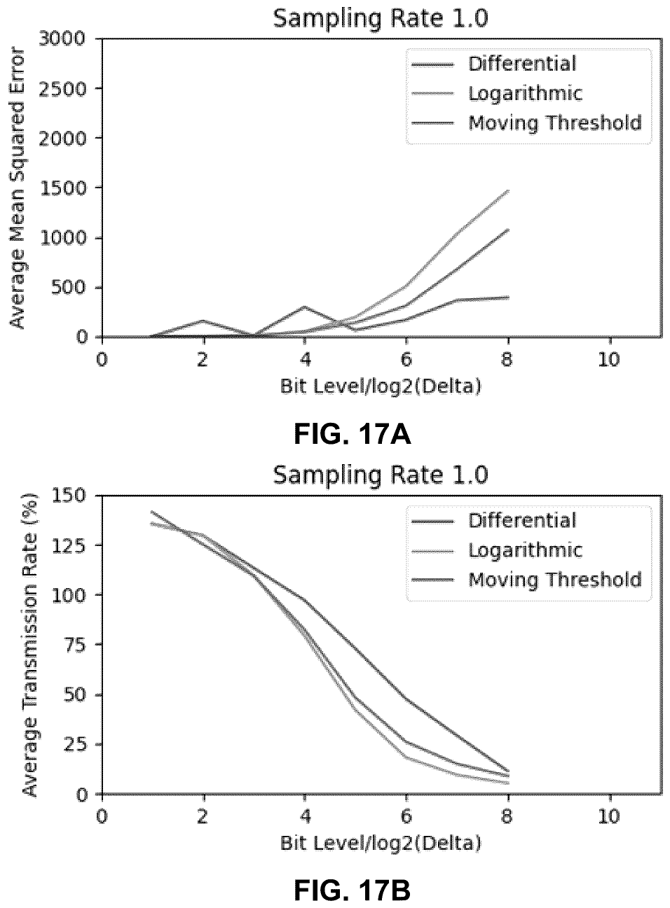

FIGS. 17A and 17B illustrate the average mean squared error and average transmission rate (%) versus Bit Level/log2 (Delta) using a sampling rate of 1.0 for the Differential, Logarithmic, and Moving Threshold algorithms.

The result indicate LOGARITHMIC performs surprisingly well.

### Analysis of Event-Driven Sampling Algorithms

Best image quality: DIFFERENTIAL algorithm, second worst performance in terms of data rate reduction.
Lowest sampling rate: QUADRATURE_PHASE algorithm, but very poor image quality.
Combination: MOVING_THRESHOLD algorithm: 3rd best data rate reduction, 2nd best image quality.
Combination with reduced sampling rate: LOGARITHMIC algorithm, at 60% of original sampling frequency lowest transmission rate (<25% on average) and similar quality to DIFFERENTIAL.
These results may change with different imaging modes.
Use may be dependent on which is easiest to implement in hardware.
   Comparison with MicroBeamforming
Algorithms Emulation Results show results for MicroBeamforming and other ideas to reduce data rate.
Microbeamforming of 2 channels (a flat 50% data rate reduction) achieved MSEs of 60-100 for 3 different datasets (single frame)-this has the advantage of a deterministic data rate rather than a variable one as with these event-driven sampling architectures.
The microbeamforming achieved metrics for the 2022-07-28 _IMMR pullback analyzed previously of: 50% transmission rate/157 MSE/26 dB PSNR/0.126 NRMSE
   Plots of Average Transmission over all events

The following are plots of average transmission rate over all events (error bars show min and max transmission), mean squared error, PSNR and NRMSE for the three most promising event-driven algorithms. The plots were truncated at 200 MSE to show when this threshold was crossed, as this performance would be worse than microbeamforming.

Data related to 2022-09-8 012_VeraStudy Snapshot 14 Frame 30 (Preclinical)

Microbeamforming (n=2): 50% data transmission, 65.9 MSE/30 dB PSNR/0.226 NRMSE

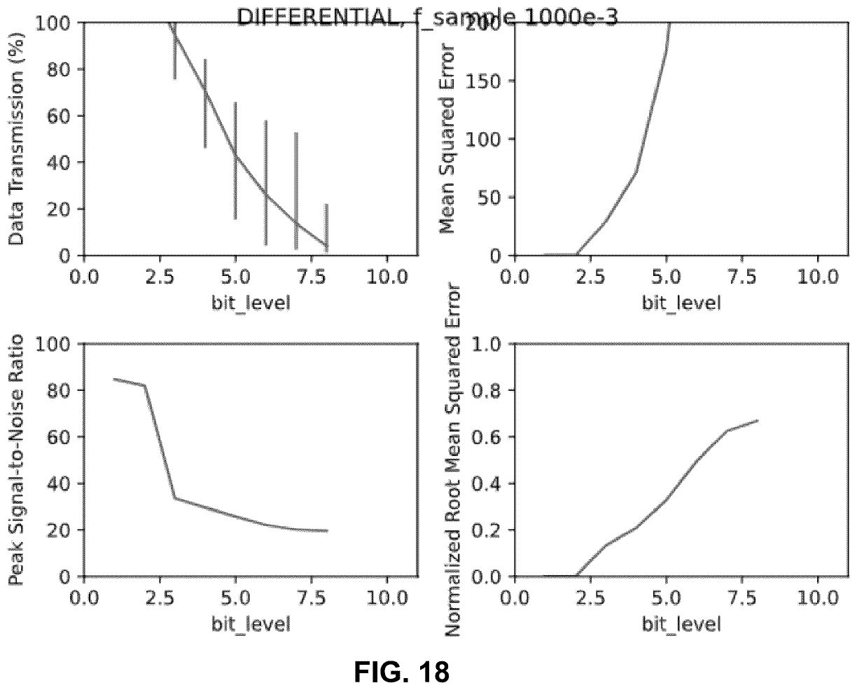

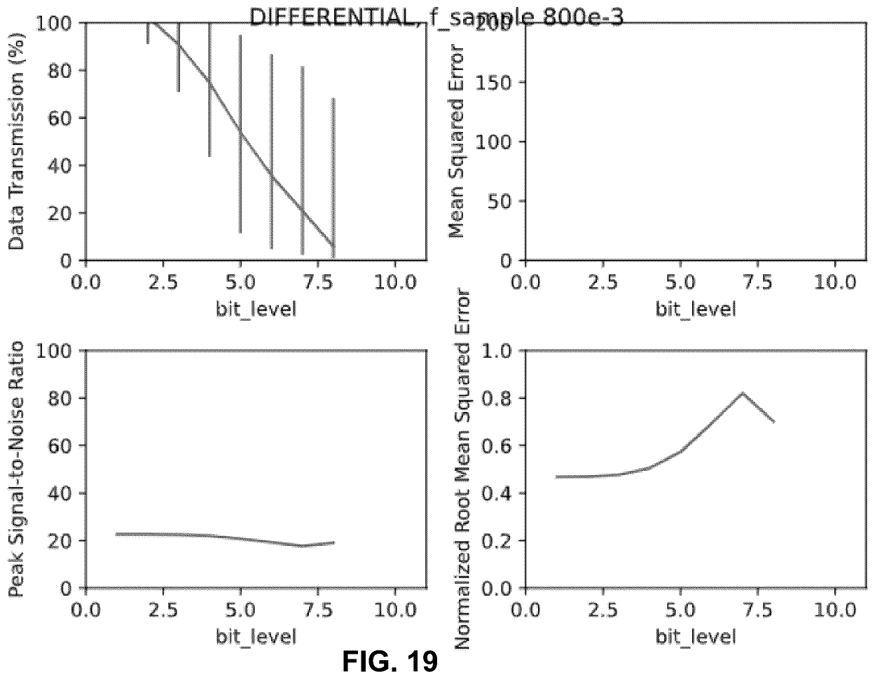

FIG. 18 includes graphs of mean data transmission rate (%), mean squared error, peak signal-to-noise ratio, and normalized root mean squared error versus bit_level for DIFFERENTIAL_f_sample_1000e-3.

FIG. 19 includes graphs of mean data transmission rate (%), mean squared error, peak signal-to-noise ratio, and normalized root mean squared error versus bit_level for DIFFERENTIAL_f_sample_800e-3.

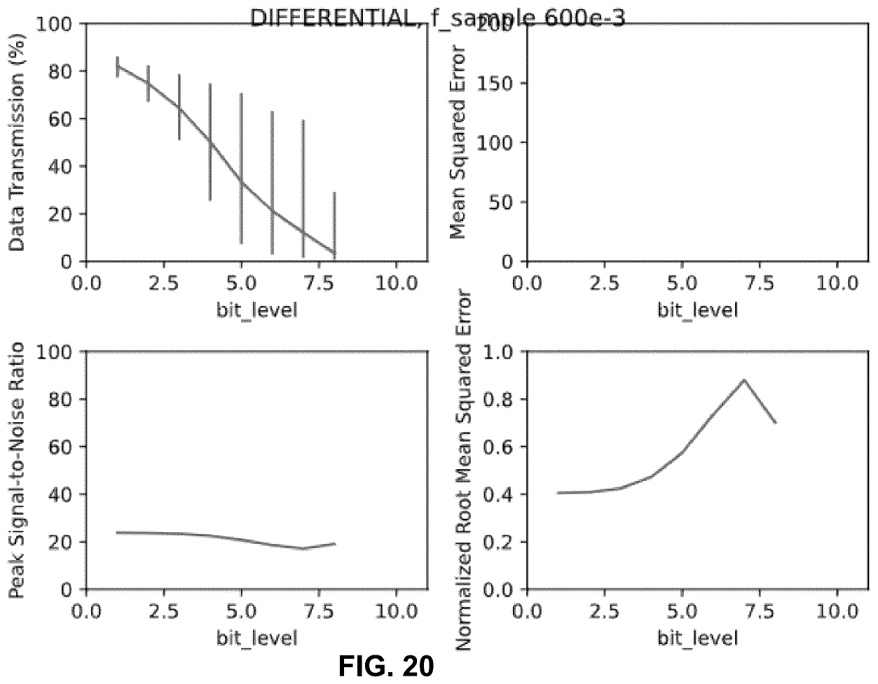

FIG. 20 includes graphs of mean data transmission rate (%), mean squared error, peak signal-to-noise ratio, and normalized root mean squared error versus bit_level for DIFFERENTIAL_f_sample_600e-3.

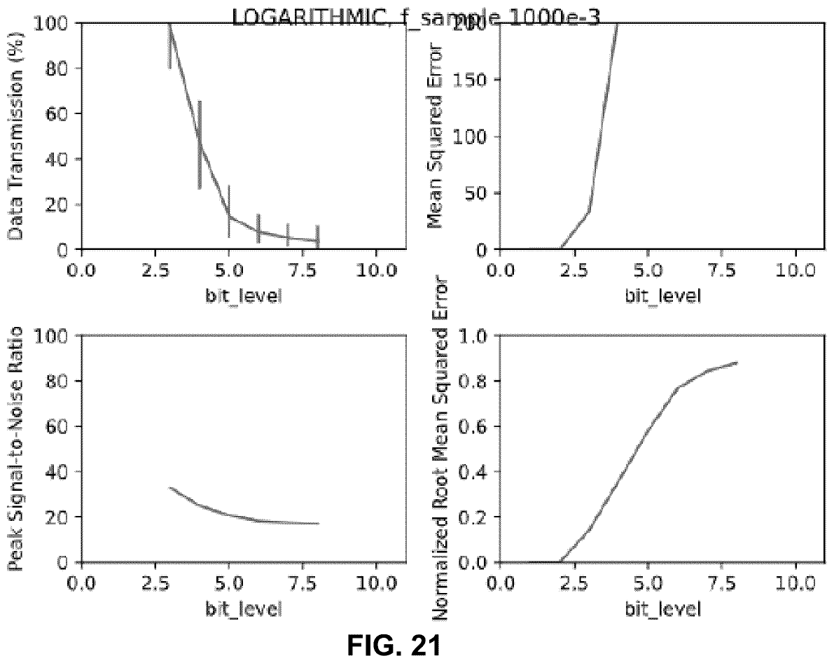

FIG. 21 includes graphs of mean data transmission rate (%), mean squared error, peak signal-to-noise ratio, and normalized root mean squared error versus bit_level for LOGARITHMIC_f_sample_1000e-3.

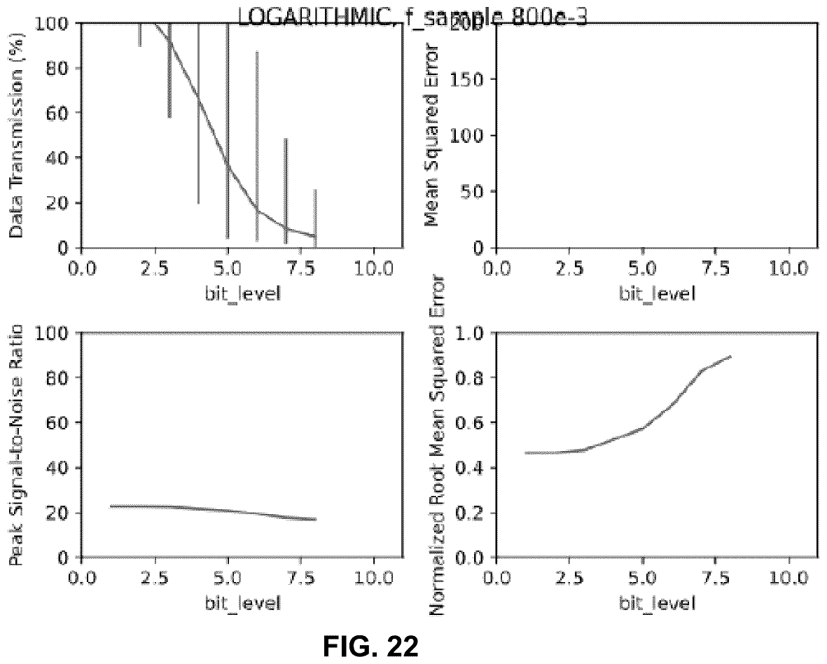

FIG. 22 includes graphs of mean data transmission rate (%), mean squared error, peak signal-to-noise ratio, and normalized root mean squared error versus bit_level for LOGARITHMIC_f_sample_800e-3.

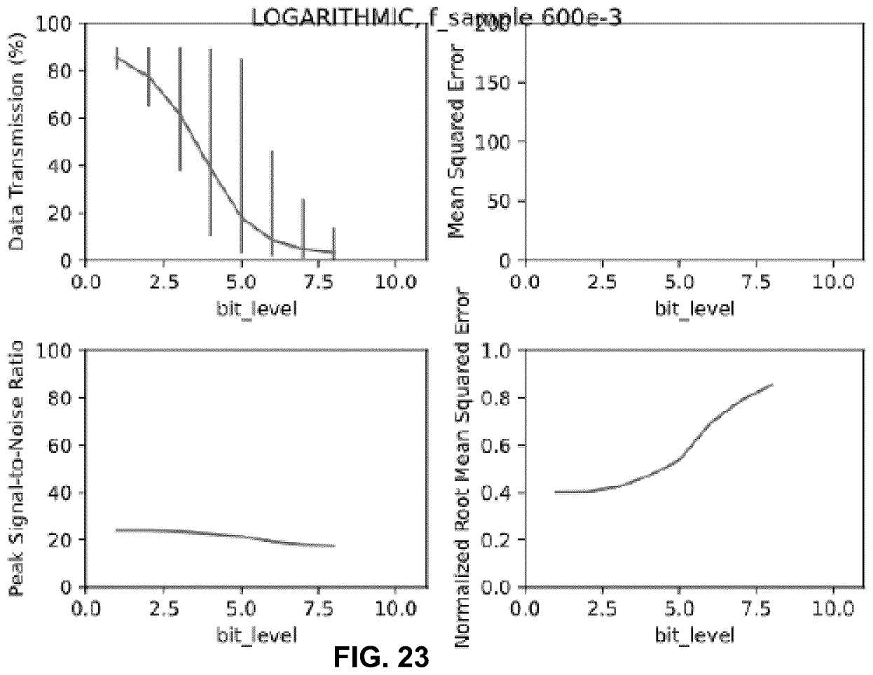

FIG. 23 includes graphs of mean data transmission rate (%), mean squared error, peak signal-to-noise ratio, and normalized root mean squared error versus bit_level for LOGARITHMIC_f_sample_600e-3.

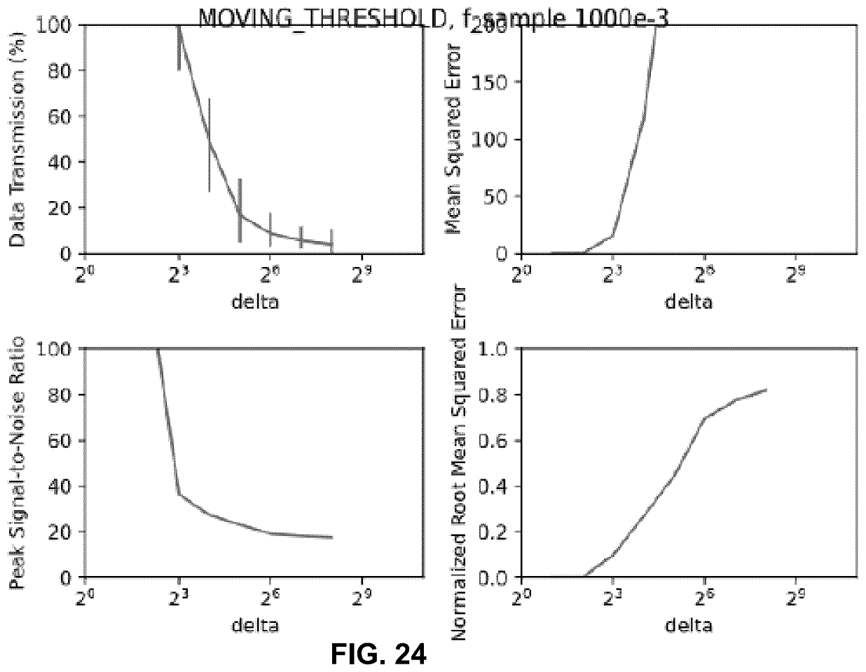

FIG. 24 includes graphs of mean data transmission rate (%), mean squared error, peak signal-to-noise ratio, and normalized root mean squared error versus delta (bit_level) for MOVING_THRESHOLD_f_sample_1000e-3.

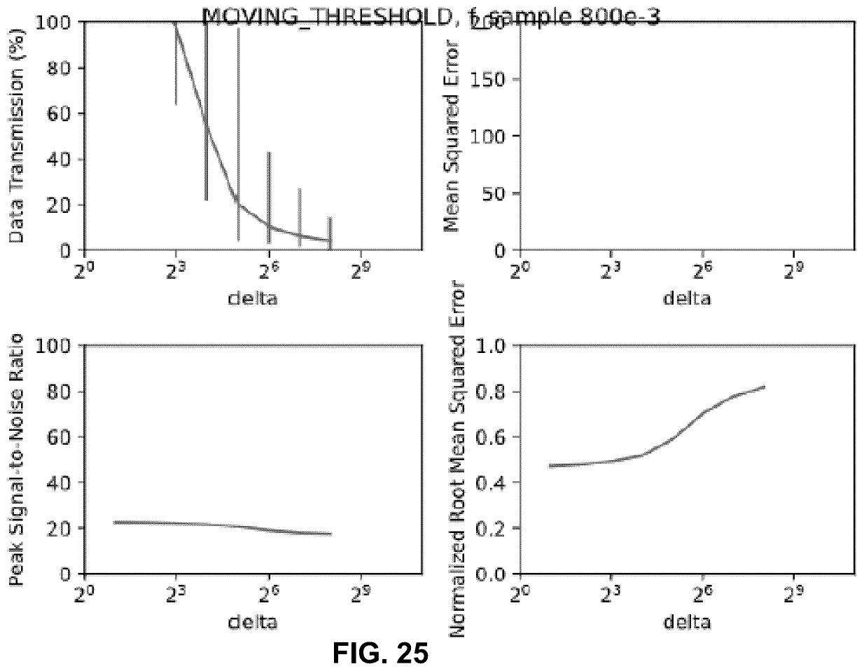

FIG. 25 includes graphs of mean data transmission rate (%), mean squared error, peak signal-to-noise ratio, and normalized root mean squared error versus delta (bit_level) for MOVING_THRESHOLD_f_sample_800e-3.

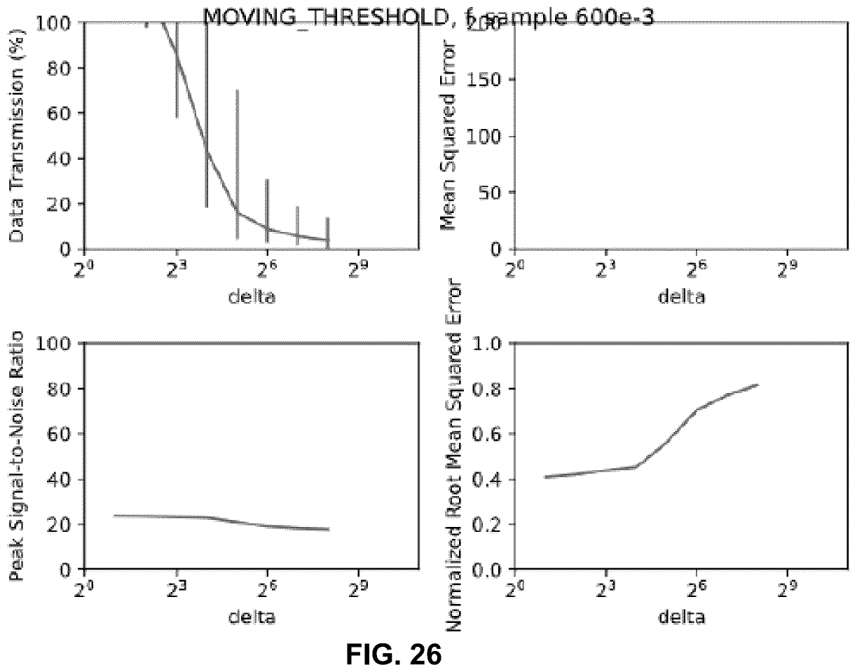

FIG. 26 includes graphs of mean data transmission rate (%), mean squared error, peak signal-to-noise ratio, and normalized root mean squared error versus delta (bit_level) for MOVING_THRESHOLD_f_sample_600e-3.

Plots of Average Transmission over all events

The following are plots of average transmission rate over all events (error bars show min and max transmission), mean squared error, PSNR and NRMSE for the three most promising event-driven algorithms. The plots were truncated at 200 MSE to show when this threshold was crossed, as this performance would be worse than microbeamforming.

Data related to 2022-10-18 000_Verastudy Snapshot 00 Frame 25 (CIRS Phantom)

Microbeamforming (n=2): 50% data transmission, 113 MSE/28 dB PSNR/0.0186 NRMSE

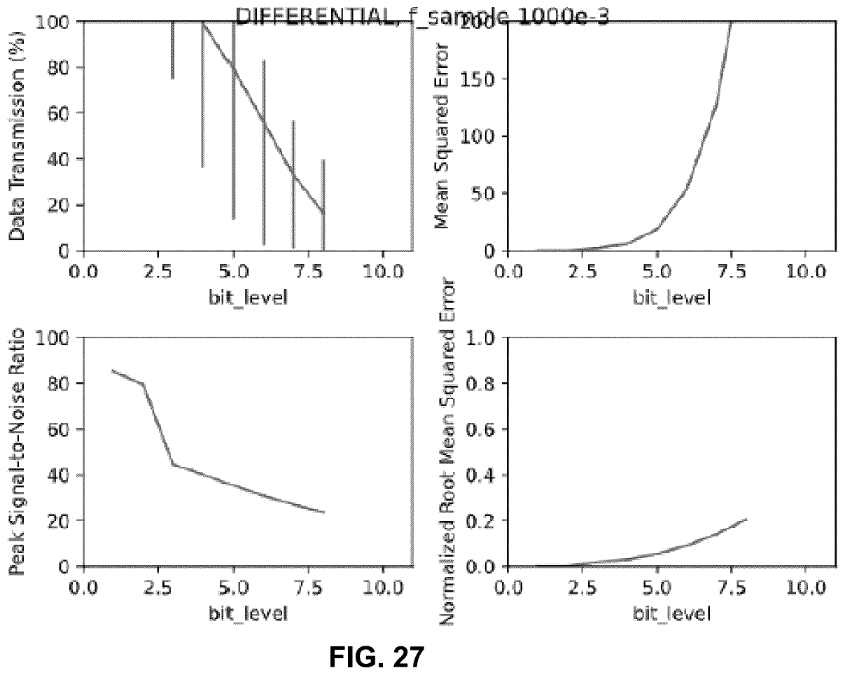

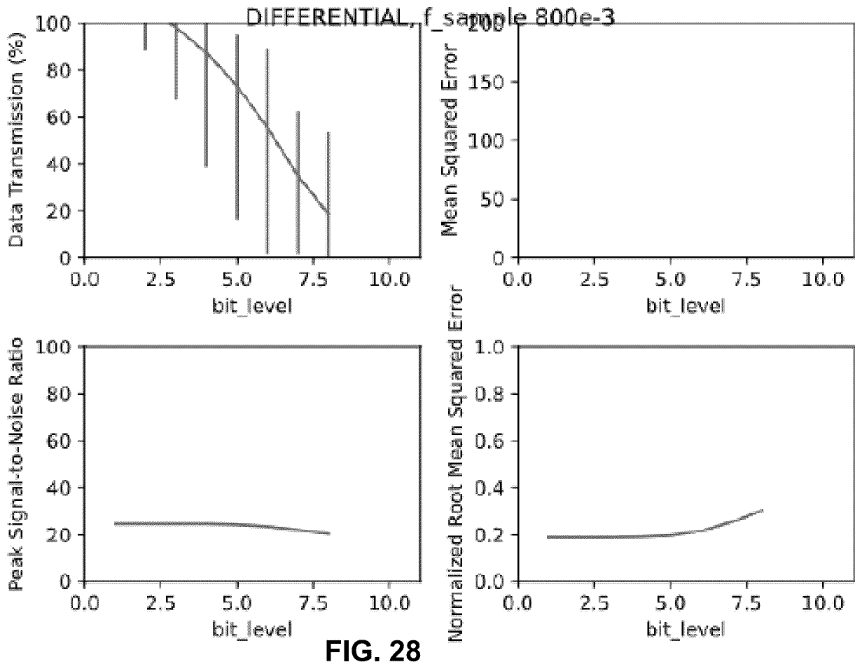

FIG. 27 includes graphs of mean data transmission rate (%), mean squared error, peak signal-to-noise ratio, and normalized root mean squared error versus bit_level for DIFFERENTIAL_f_sample_1000e-3.

FIG. 28 includes graphs of mean data transmission rate (%), mean squared error, peak signal-to-noise ratio, and normalized root mean squared error versus bit_level for DIFFERENTIAL_f_sample_800e-3.

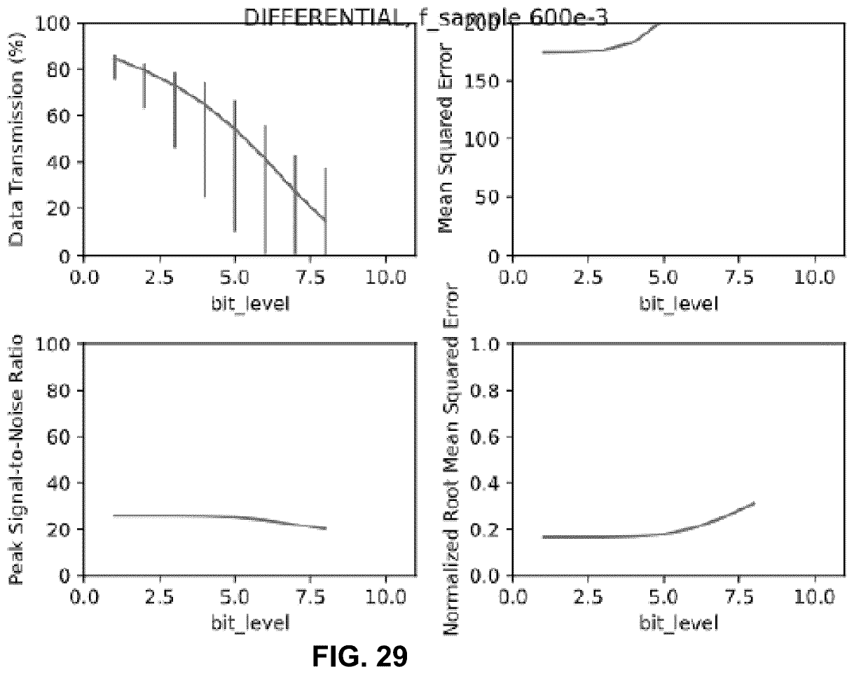

FIG. 29 includes graphs of mean data transmission rate (%), mean squared error, peak signal-to-noise ratio, and normalized root mean squared error versus bit_level for DIFFERENTIAL_f_sample_600e-3.

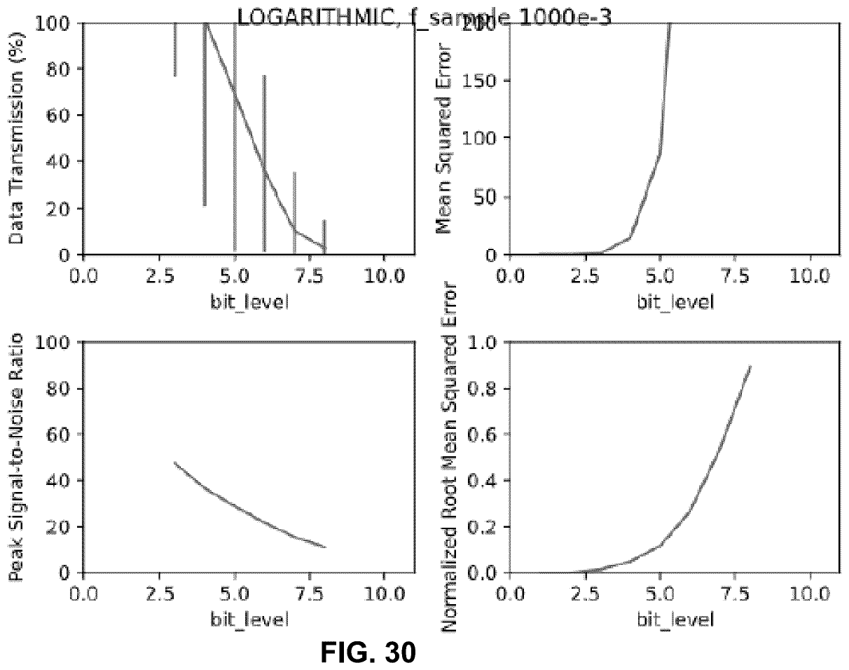

FIG. 30 includes graphs of mean data transmission rate (%), mean squared error, peak signal-to-noise ratio, and normalized root mean squared error versus bit_level for LOGARITHMIC_f_sample_1000e-3.

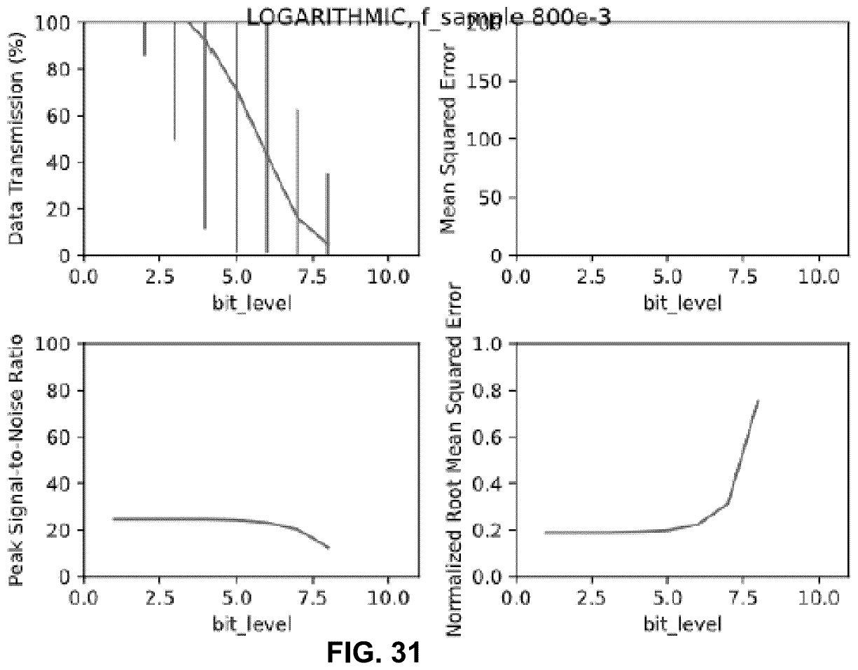

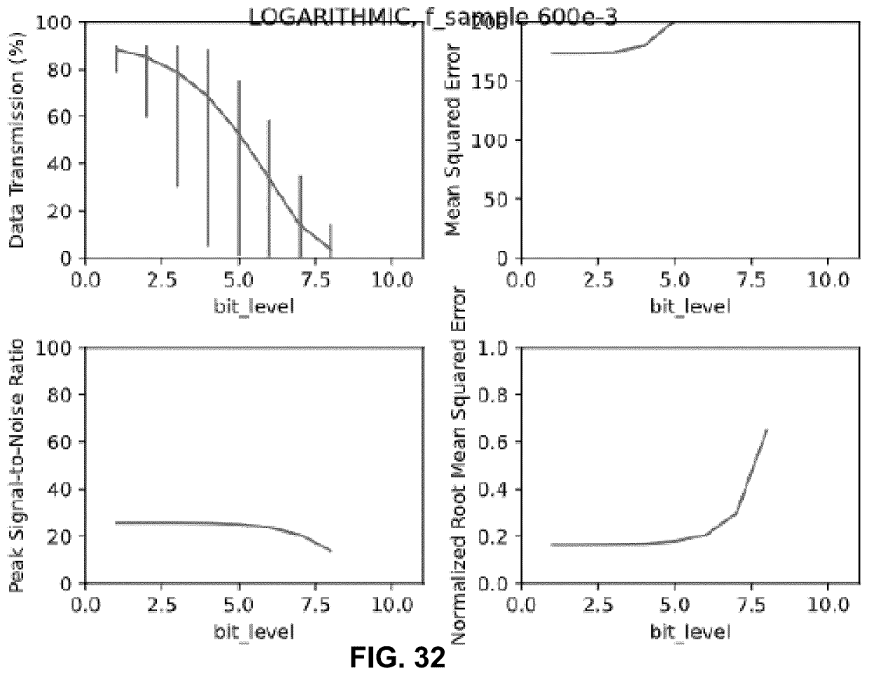

FIG. 31 includes graphs of mean data transmission rate (%), mean squared error, peak signal-to-noise ratio, and normalized root mean squared error versus bit_level for LOGARITHMIC_f_sample_800e-3.

FIG. 32 includes graphs of mean data transmission rate (%), mean squared error, peak signal-to-noise ratio, and normalized root mean squared error versus bit_level for LOGARITHMIC_f_sample_600e-3.

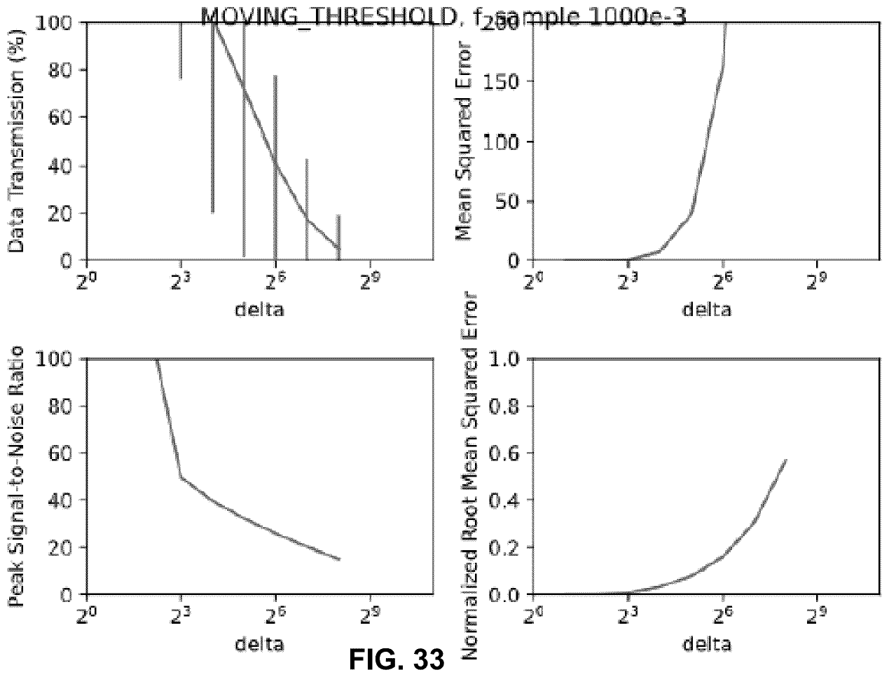

FIG. 33 includes graphs of mean data transmission rate (%), mean squared error, peak signal-to-noise ratio, and normalized root mean squared error versus delta (bit_level) for

MOVING_THRESHOLD_f_sample_1000e-3.

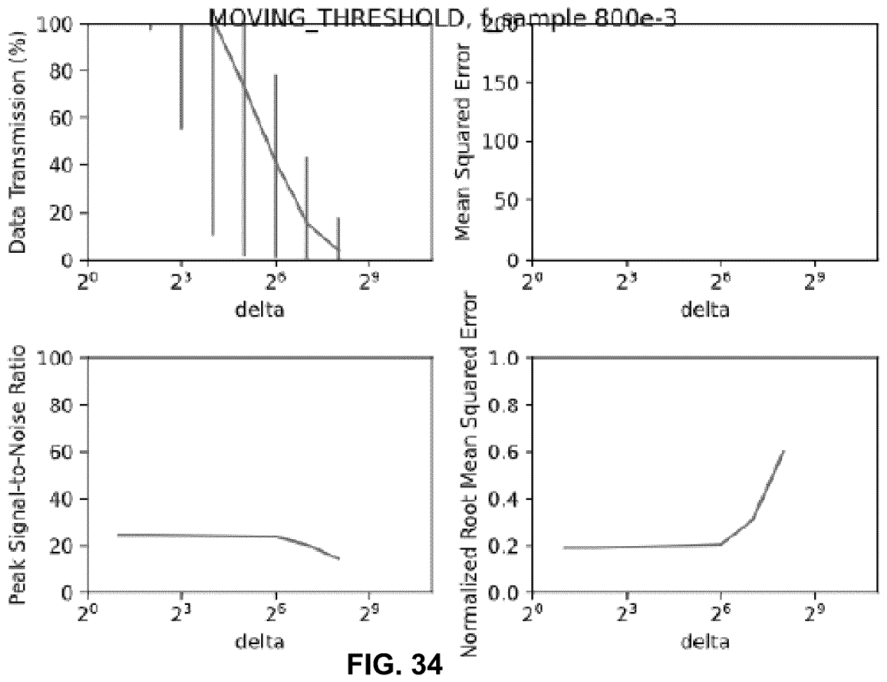

FIG. 34 includes graphs of mean data transmission rate (%), mean squared error, peak signal-to-noise ratio, and normalized root mean squared error versus delta (bit_level) for MOVING_THRESHOLD_f_sample_800e-3.

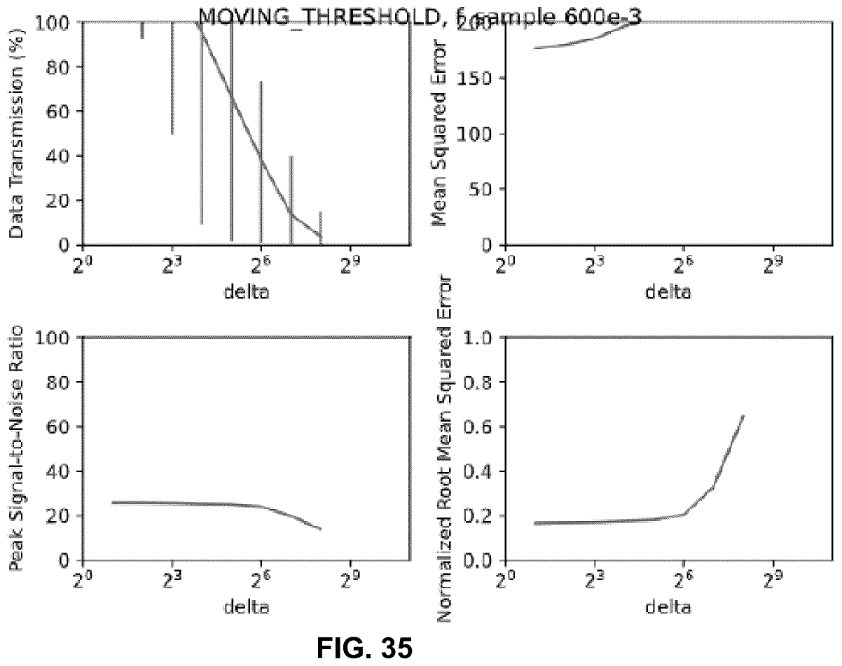

FIG. 35 includes graphs of mean data transmission rate (%), mean squared error, peak signal-to-noise ratio, and normalized root mean squared error versus delta (bit_level) for MOVING_THRESHOLD_f_sample_600e-3.

Results suggest that the moving threshold algorithm offers the best combination of reduced data rate (<30% data rate averaged over all frames) and image quality (measured in terms of the mean-squared error of beamformed images relative to full sampling).

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for image sampling, the system comprising a hardware processor coupled to non-transitory, computer-readable memory containing instructions executable by the processor to cause the processor to receive data from an imaging device and run an event-driven sampling algorithm causing the processor to:
analyze the received data to identify one or more characteristics associated with the received data and further detect the occurrence of one or more events, wherein the one or more events comprises a change in the one or more characteristics as compared to a defined threshold for the one or more characteristic; and
tune a sampling rate of the imaging device based on a detected occurrence of the one or more events.

2. The system of claim 1, wherein the defined threshold is determined based, at least in part, on a clinical relevance of a structure to be imaged and/or one or more characteristics of the received data.

3. The system of claim 1, wherein the received data comprises analog voltage signals.

4. The system of claim 1, wherein the received data comprises digitized voltage signals.

5. The system of claim 4, wherein the processor is configured to:
access raw digitized voltage signals; and
tune one or more sampling parameters to minimize a data transmission rate and to maximize an image quality of a clinically relevant image.

6. The system of claim 4, wherein the defined threshold is a digitized voltage encoded at a lower bit depth than a full sampling bit depth.

7. The system of claim 4, wherein the defined threshold is a change in output voltage by a defined parameter relative to a previous output voltage.

8. The system of claim 7, wherein image sampling by the imaging device is triggered at a full sampling rate for a set time for each occurrence of an event associated with the output voltage changing by the defined parameter relative to the previous output voltage.

9. The system of claim 4, wherein the defined threshold is a change in output voltage by a set parameter in a previously stored threshold value, wherein the threshold value is updated and stored when image sampling by the imaging device is triggered.

10. The system of claim 4, wherein the defined threshold is one or more logarithmically spaced voltage levels.

11. The system of claim 1, wherein the defined threshold is a change in an amplitude signal by a set parameter in a previously stored threshold amplitude signal, wherein only amplitude and phase of a carrier wave are sampled with the phase sampled concurrently with the amplitude and encoded at a lower bit rate than 16 bits.

12. The system of claim 11, wherein the amplitude and the phase of the carrier wave are extracted via I/Q demodulation.

13. The system of claim 1, wherein the imaging device comprises a transducer comprising an array of individual imaging elements.

14. The system of claim 13, wherein the transducer comprises a micro-electromechanical systems (MEMS)-based micromachined ultrasonic transducer configured as a two-dimensional (2D) array structure.

15. The system of claim 13, wherein the imaging elements are acoustic sensors activated by the processor to transmit and/or receive a plurality of incident acoustic wave signals as wave data.

16. The system of claim 15, wherein the wave data comprises at least one of plane wave data and diverging wave data associated with one or more wave transmit-receive cycles carried out by the imaging elements.

17. The system of claim 16, wherein the wave data is full circumferential, three-dimensional (3D) image data.

18. The system of claim 17, wherein the imaging device comprises a catheter-based ultrasound imaging device configured to transmit ultrasound pulses to, and receive echoes of the ultrasound pulses from, intravascular and/or intracardiac tissue.

19. The system of claim 13, wherein the imaging device is a minimally invasive implantable device.

20. The system of claim 19, wherein tuning of the sampling rate reduces an overall power consumption and heat dissipation of the device.

21. The system of claim 1, wherein tuning of the sampling rate results in reduction of an average sampling rate of received data from the imaging device.

22. The system of claim 1, wherein the processor is embedded as part of an application-specific integrated circuit (ASIC).
